SEQUENCE LISTING (7 sequences)

SEQ ID NO: 1 - DNA, 25 bp, synthetic construct
  source 1..25: mol_type = other DNA
caccgtttctcatagcgaccaccag

SEQ ID NO: 2 - DNA, 25 bp, synthetic construct
  source 1..25: mol_type = other DNA
aaacctggtggtcgctatgagaaac

SEQ ID NO: 3 - DNA, 24 bp, synthetic construct
  source 1..24: mol_type = other DNA
caccgcgtgctccgttcgcgcttc

SEQ ID NO: 4 - DNA, 24 bp, synthetic construct
  source 1..24: mol_type = other DNA
aaacgaagcgcgaacggagcacgc

SEQ ID NO: 5 - DNA, 20 bp, synthetic construct
  source 1..20: mol_type = other DNA
ggctggaaggtgacacagtt

SEQ ID NO: 6 - DNA, 20 bp, synthetic construct
  source 1..20: mol_type = other DNA
cccaaaaccaacaccaactc

SEQ ID NO: 7 - DNA, 20 bp, synthetic construct
  source 1..20: mol_type = other DNA
gacacagttcttgccacagc

(19) United States
(12) Patent Application Publication
(10) Pub. No.: US 2024/0239901 A1
(43) Pub. Date: Jul. 18, 2024

(54) Methods and Compositions for Treating Mucus Obstruction in Severe Asthma

(71) Applicant: National Jewish Health, Denver, CO (US)

(72) Inventors: Max A. Seibold, Denver, CO (US); Jamie L. Everman, Denver, CO (US)

(21) Appl. No.: 18/412,165

(22) Filed: Jan. 12, 2024

Related U.S. Application Data
(60) Provisional application No. 63/479,685, filed on Jan. 12, 2023.

(51) Int. Cl. C07K 16/28

(52) U.S. Cl. CPC C07K 16/2851

(57) ABSTRACT

Disclosed herein are methods/uses as well as compositions for reducing airway mucus obstruction, mucus plugging and/or airway inflammation by administering to a subject in need thereof, an intelectin-1 (ITLN-1) inhibitor.

### CROSS REFERENCE TO RELATED APPLICATIONS

This application claims the benefit of priority under 35 U.S.C. § 119(e) to U.S. Provisional Patent Application No. 63/479,685, filed Jan. 12, 2023. The entire disclosure of U.S. Provisional Patent Application No. 63/479,685 is incorporated herein by reference.

### GOVERNMENT SUPPORT CLAUSE

This invention was made with government support under Grant Nos. R01HL135156, P01HL107202, R01HL128439 and P01HL132821, each awarded by the National Institutes of Health. The government has certain rights in the invention.

### REFERENCE TO A SEQUENCE LISTING

This application contains a Sequence Listing submitted electronically as an ST.26 XML file. The XML file, named “2879-242.xml”, has a size of 8000 Bytes, and was recorded on Jan. 12, 2024. The information contained in the XML file is incorporated herein by reference in its entirety.

### BACKGROUND

Airway inflammation results in overproduction of mucus into the small and large airways. This often results in occlusion and blockage of the airways, resulting in extremely difficult breathing, and impacts downstream functional properties of the airways (protection from pathogens, gas exchange, etc). Development of mucolytic agents (drugs that target mucin proteins which are a key protein in mucus plugs) has been slow and often involve chemicals that have toxic side-effects. To date, the structure of airway mucus has been poorly characterized, and very few non-mucin molecules have been identified as potential biological targets to combat mucus plugging and obstruction.

Airway molecular characterization (endotyping) studies have revealed significant pathobiological heterogeneity among patients with asthma (Fahy, J.V. Type 2 inflammation in asthma—present in most, absent in many. Nat Rev Immunol 15, 57-65 (2015); Wesolowska-Andersen, A. & Seibold, M. A. Airway molecular endotypes of asthma: dissecting the heterogeneity. Curr Opin Allergy Clin Immunol 15, 163-168 (2015)). The most prevalent asthma endotype involves inflammation of the airways driven by type 2 (T2) cytokines, including interleukins 4, 5, and 13 (IL-4, IL-5, IL-13) (Woodruff, P. G., et al. T-helper type 2-driven inflammation defines major subphenotypes of asthma. Am J Respir Crit Care Med 180, 388-395 (2009); Sajuthi, S. P., et al. Type 2 and interferon inflammation regulate SARS-CoV-2 entry factor expression in the airway epithelium. Nat Commun 11, 5139 (2020); Jackson, N. D., et al. Single-Cell and Population Transcriptomics Reveal Pan-epithelial Remodeling in Type 2-High Asthma. Cell Rep 32, 107872 (2020); Peters, M. C., et al. A Transcriptomic Method to Determine Airway Immune Dysfunction in T2-High and T2-Low Asthma. Am J Respir Crit Care Med 199, 465-477 (2019); Poole, A., et al. Dissecting childhood asthma with nasal transcriptomics distinguishes subphenotypes of disease. J Allergy Clin Immunol 133, 670-678 e612 (2014)). Airway T2 inflammation is associated with more severe airflow obstruction, which may result from pathologic mucus production, leading to airway plugging. Indeed, computed tomography (CT) lung scans from patients with asthma show a high prevalence of airway mucus plugs, which correlate strongly with measures of airway T2 inflammation and airflow obstruction (Dunican, E. M., et al. Mucus plugs in patients with asthma linked to eosinophilia and airflow obstruction. J Clin Invest 128, 997-1009 (2018)). Supporting the involvement of T2 inflammation in mucus dysfunction, in vitro analyses of human airway epithelial cell cultures have uncovered that IL-13 triggers mucus hypersecretion and the metaplastic generation of mucus secretory cells, which produce a pathologic mucus that arrests mucociliary movement (Jackson, N. D., et al. Single-Cell and Population Transcriptomics Reveal Pan-epithelial Remodeling in Type 2-High Asthma. Cell Rep 32, 107872 (2020); Bonser, L. R., Zlock, L., Finkbeiner, W. & Erle, D. J. Epithelial tethering of MUC5AC-rich mucus impairs mucociliary transport in asthma. J Clin Invest 126, 2367-2371 (2016)).

The mechanisms underlying the formation of pathologic, T2 inflammation-induced airway mucus are unclear, but may involve changes to the protein composition of the mucus. Clinical studies show that the normal predominance of MUC5B mucin in healthy airway mucus is shifted towards higher expression of MUC5AC in asthma (Lachowicz-Scroggins, M. E., et al. Abnormalities in MUC5AC and MUC5B Protein in Airway Mucus in Asthma. Am J Respir Crit Care Med 194, 1296-1299 (2016)). Supporting a pathologic role for MUC5AC, a recent study found that MUC5AC protein is poorly extruded from the epithelium and can tether the mucus gel to the epithelium, limiting its mucociliary movement (Bonser, L. R., Zlock, L., Finkbeiner, W. & Erle, D. J. Epithelial tethering of MUC5AC-rich mucus impairs mucociliary transport in asthma. J Clin Invest 126, 2367-2371 (2016)). The potential for other protein components of mucus to be involved in T2-related mucus pathology was suggested by our recent study finding that IL-13 stimulated mucociliary airway epithelial cultures secrete a unique repertoire of proteins (Jackson, N. D., et al. Single-Cell and Population Transcriptomics Reveal Pan-epithelial Remodeling in Type 2-High Asthma. Cell Rep 32, 107872 (2020)). Among these proteins was intelectin-1 (ITLN-1), a lectin which can bind galactosyl glycans expressed by microbes but does not bind to mammalian glycans (Wesener, D. A., et al. Recognition of microbial glycans by human intelectin-1. Nat Struct Mol Biol 22, 603-610 (2015); Tsuji, S., et al. Human intelectin is a novel soluble lectin that recognizes galactofuranose in carbohydrate chains of bacterial cell wall. J Biol Chem 276, 23456-23463 (2001)). Importantly, ITLN-1 has also been reported to be a prominent protein constituent of mucus plugs in fatal asthma, although its role in airway mucus pathology is unknown (Kerr, S. C., et al. Intelectin-1 is a prominent protein constituent of pathologic mucus associated with eosinophilic airway inflammation in asthma. Am J Respir Crit Care Med 189, 1005-1007 (2014)). Notably, a genetic variant at the ITLN1 locus is associated with both Crohn's disease and asthma suggesting a role in inflammatory mucosal diseases (Pemberton, A. D., Rose-Zerilli, M. J., Holloway, J. W., Gray, R. D. & Holgate, S. T. A single-nucleotide polymorphism in intelectin 1 is associated with increased asthma risk. J Allergy Clin Immunol 122, 1033-1034 (2008); Barrett, J. C., et al. Genome-wide association defines more than 30 distinct susceptibility loci for Crohn's disease. Nat Genet 40, 955-962 (2008); Franke, A., et al. Genome-wide meta-analysis increases to 71 the number of confirmed Crohn's disease susceptibility loci. Nat Genet 42, 1118-1125 (2010)).

As disclosed herein, by using scRNA-seq and CRISPR gene editing of human mucociliary airway epithelia, the role of ITLN-1 protein in IL-13-induced mucostasis is defined, and the inventors demonstrate the ability of ITLN-1 to bind airway mucins via non-lectin based electrostatic interactions. In asthma cohort studies in children and adults, the genetic and inflammatory regulators of ITLN1 expression are defined herein and the inventors show how functional genetic variation in the ITLN1 gene influences susceptibility to airway mucus plugging.

### SUMMARY

One embodiment relates to a method and/or use of reducing mucus obstruction and/or airway inflammation in a subject in need thereof, comprising administering to the subject an intelectin-1 (ITLN-1) inhibitor.

In one aspect of any of the methods and/or uses disclosed herein, the subject is a T2-high asthmatic.

In one aspect of any of the methods and/or uses disclosed herein, the mucus obstruction is mucus plugging.

In one aspect of any of the methods and/or uses disclosed herein, the ITLN-1 inhibitor reduces mucus viscosity.

In one aspect of any of the methods and/or uses disclosed herein, the ITLN-1 inhibitor increases muco-ciliary clearance.

In one aspect of any of the methods and/or uses disclosed herein, the ITLN-1 inhibitor is a chemical inhibitor of ITLN-1 or a biological inhibitor of ITLN-1.

In one aspect of any of the methods and/or uses disclosed herein, the ITLN-1 inhibitor is an anti-intelectin molecule or an anti-intelectin antibody.

In one aspect of any of the methods and/or uses disclosed herein, the ITLN-1 inhibitor is in a pharmaceutical composition.

Another embodiment relates to a composition comprising an ITLN-1 inhibitor.

In one aspect of any of the compositions disclosed herein, the ITLN-1 inhibitor reduces mucus obstruction in a T2-high asthmatic subject.

In one aspect of any of the compositions disclosed herein, the mucus obstruction is mucus plugging.

In one aspect of any of the compositions disclosed herein, the ITLN-1 inhibitor reduces mucus viscosity.

In one aspect of any of the compositions disclosed herein, the ITLN-1 inhibitor increases muco-ciliary clearance.

In one aspect of any of the compositions disclosed herein, the ITLN-1 inhibitor is a chemical inhibitor of ITLN-1 or a biological inhibitor of ITLN-1.

In one aspect of any of the compositions disclosed herein, the ITLN-1 inhibitor is an anti-intelectin molecule or an anti-intelectin antibody.

One embodiment relates to a pharmaceutical composition comprising an ITLN-1 inhibitor, for use in reducing mucus obstruction in a subject in need thereof.

One embodiment relates to the use of the pharmaceutical compositions disclosed herein for use in a method of reducing mucus obstruction in a subject in need thereof.

One embodiment relates to the use of the pharmaceutical compositions disclosed herein for the manufacture of a medicament for reducing mucus obstruction in a subject in need thereof.

### BRIEF DESCRIPTION OF THE DRAWINGS

FIGS. 1A, 1B, 1C, 1D, 1E and 1F show ITLN-1 is an IL-13-induced component of the airway epithelium. FIG. 1A shows a chronic IL-13 stimulation model of airway epithelial T2-inflammation as illustrated by H&E staining and immunofluorescent (IF) labeling of MUC5AC (red), MUC5B (green), and nuclei (blue); images are representative of 8 paired donors analyzed; scale bar 30 uM. FIG. 1B shows a box plot of normalized ITLN1 gene expression in baseline (control) and IL-13 treated ALI cultures (n=19 donors). FDR-adjusted two-sided p-value is based on a paired exact test (edgeR). FIG. 1C shows enrichment of ITLN1 co-expression network genes in mucus secretory cells, compared to all other defined airway epithelial lung cell populations identified by scRNA-seq. FDR-adjusted two-sided p-values are based on a one-sided hypergeometric test. FIG. 1D shows immunofluorescent labeling showing IL-13-induced expression of ITLN-1 in a subset of MUC5AC⁺ secretory cells; MUC5AC (red), ITLN-1 (green), nuclei (blue); images are representative of 8 paired donors analyzed; scale bar 30 uM. FIG. 1E shows box plots of normalized ITLN-1 peptides measured in apical ALI secretions following control and chronic IL-13 stimulation (aqueous fraction n=14 donors, mucus fraction n=9 donors). FDR-adjusted two-sided p-values are based on a paired exact test (edgeR). LFC=log fold change. FIG. 1F shows representative images from confocal imaging analysis mucociliary ALI cultures stimulated with IL-13 illustrating ITLN-1 presence within the airway mucus on the apical side of mucociliary epithelium; MUC5AC (red), ITLN-1 (green), F-actin (white); black arrows—apical cell membrane, yellow arrows—mucus layer; XY image scale bar 10 μM.

FIGS. 2A, 2B, 2C and 2D show ITLN-1 contributes to IL-13-mediated decrease in epithelial mucociliary function. FIG. 2A is a schematic of the gene structure of ITLN1, the targeted CRISPR-Cas9 editing site, and the resultant DNA editing as determined by high resolution melt curve analysis. The plot (bottom) shows differences in melt temperature profiles from pooled ALI inserts (n=3/condition) for ITLN1 KO (red) samples from each donor assayed by HRM analysis. The scramble control DNA melt temperature profile (blue) was set as the reference for each donor (n=3 donors). FIG. 2B is a western blot quantitation of ITLN-1 (35 kDa) in apical washes collected from mock or IL-13-stimulated mucociliary ALI cultures differentiated from control edited (scrb) and ITLN1 KO basal cells; Western blot image is representative of all measured data, and band intensity plot reflects data from conditions from all edited donors (n=3); Two sided p-values calculated using Student's t-test ***p<0.001. FIG. 2C are box plots showing the log of average particle speed within control edited (scrb) and ITLN1 KO mucociliary cultures that were mock-(BSA, white boxes) or IL-13-stimulated (grey boxes), with replicate experiments carried out using no wash, PBS wash, PBS-DTT wash, and ATP+PBS-DTT wash regimes. Average speed values are colored by donor, where there are 18 datapoints for each condition-donor pair (6 fields of view×3 inserts). Above plots are given the estimated percent recovery (and associated p-values) of particle speed in IL-13-stimulated cultures relative to BSA cultures when in an ITLN1 KO epithelium compared to the control (scrb) epithelium. Two-sided p-values are based on linear mixed model t-tests, with random effects for donor and insert, and use Satterthwaite approximation of degrees of freedom. FIG. 2D show box plots of ciliary beat frequency (CBF) measured on control (scrb) and ITLN1 KO mucociliary ALI cultures from triplicate culture inserts and 3 donors following mock and IL-13 stimulation. Each box plot represents all datapoints collected from all videos captured across donors and inserts for each condition (N=30,899, 24,786, 25,533, and 24,964, from left to right), but illustration of outliers was excluded for visualization purposes. Two-sided p-values are based on linear mixed model t-tests with random effects for donor and insert and use Satterthwaite approximation of degrees of freedom.

FIGS. 3A, 3B and 3C show ITLN-1 binds to purified airway mucins and to the C-terminal domain of MUC5AC. FIG. 3A is a bar graph measuring binding of recombinant flag-tagged ITLN-1 protein, and the effect of co-incubation with galactose, heparin, or dextran sulfate, to purified human airway mucins (isolated and pooled from n=5 subjects); one way ANOVA ** p<0.01, *** p<0.001 and **** p<0.0001. FIG. 3B shows binding assays measuring the ability of ITLN-1 protein, or co-incubated ITLN-1+heparin, to bind to immobilized C-terminal peptides of MUC5AC (left) or MUC5B (right); two-sided t-test **** p<0.0001, ns=not significant. FIG. 3C is an illustration of the potential mechanism by which an ITLN-1 protein trimer could interact with the C-terminal domain of MUC5AC mucin molecules via electrostatic interactions.

FIGS. 4A, 4B, 4C, 4D, 4E and 4F show ITLN1 is correlated with T2 inflammatory mucus secretory expression networks and is expressed by mucus secretory cells in vivo. FIG. 4A WGCNA conducted using gene expression from nasal airway epithelial brushings collected from 695 children in the GALAII asthma study found a strong correlation of ITLN1 expression with T2 inflammation and mucus secretory networks. Select genes from each network, Pearson correlation between expression of ITLN1 and network eigengenes, and top functional enrichments from each network are given. Enrichment p-values were obtained from a one-sided fisher exact test implemented in Enrichr. Adjusted p-values were obtained using the Benjamini-Hochberg method. FIG. 4B are box plots of log 2-normalized ITLN1 gene expression in nasal epithelial brushes stratified by T2 inflammation status (n: T2-low=331, T2-high=364). Two-sided p-value was obtained from DESeq2. FIG. 4C UMAP visualization of 11,515 cells from scRNA-seq of two dissociated bronchial airway epithelial brushings detailing the 17 cell types identified by SNN clustering through Leiden algorithm. FIG. 4D are violin plots of log count per million (CPM)-normalized ITLN1 expression across the 17 distinct cell types identified in bronchial airway brushings. FIG. 4E are box plots of log 2-normalized ITLN1 expression stratified by the A/A (n=292), A/G (n=313), or G/G (n=76) genotypes of the top eQTL variant rs4656959. Two-sided p-values were obtained from DESeq2. FIG. 4F are box plots of log 2-normalized ITLN1 expression, stratified by T2 inflammation status and the genotypes of the top eQTL variant rs4656959 (T2-low n: A/A=141, A/G=148, G/G=35 and T2-high n: A/A=151, A/G=165, G/G=41). Two-sided p-values were obtained from DESeq2.

FIGS. 5A, 5B, 5C, 5D and 5E show ITLN1 rs4656959 variant eliminates ITLN-1 protein expression in airway epithelia. FIG. 5A normalized ITLN1 gene expression from paired HBEC ALI cultures (n=19 donors) stratified by treatment (control vs. IL-13) and by ITLN1 rs4656959 genotype for both treatments (n: A/A=9, A/G=7, and G/G=3). Two-sided p-values for indicated differences are based on an exact test (edgeR). FIG. 5B show box plots showing normalized ITLN-1 protein secretion measured in aqueous (left; n: A/A=5, A/G=6, and G/G=3) and mucus (right; n: A/A=3, A/G=3, and G/G=3) fractions from apical washes of IL-13-stimulated HBEC ALI cultures. Two-sided p-values for indicated differences are based on an exact test (edgeR). FIG. 5C normalized ITLN1 and MUC5AC expression measured by qPCR from tracheal mucociliary ALI cultures stratified by treatment (control-white or IL-13-grey) and by ITLN1 rs4656959 genotype (n=5 donors/genotype). Two-sided p-values are based on linear mixed model t-tests with donor as random intercept using Satterthwaite approximation of degrees of freedom. FIG. 5D shows western blot analysis measuring ITLN-1 in apical washes from control or IL-13-stimulated tracheal ALI cultures with the A/A or G/G genotype of rs4656959 (n=5 donors/genotype). Western blot image is representative of data from all donors and band intensity data reflect all conditions from all donors (n=5 donors/genotype). Two-sided p-values calculated using Student's t-test. FIG. 5E shows immunofluorescent labeling of IL-13-treated tracheal ALI cultures for ITLN-1 (green), MUC5AC (red) and nuclei (DAPI); images are representative of all 10 donors analyzed; scale bar 50 um.

FIGS. 6A, 6B and 6C show ITLN1 rs4656959 is associated with lower mucus plug scores in T2-high asthmatics. FIG. 6A are box plots of log 2-normalized ITLN1 expression from 249 subjects in the SARP asthma cohort stratified by T2 status and ITLN1 rs4656959 genotype; n: T2-low subjects n: A/A=43, A/G=61, G/G=13 and T2-high subjects n: A/A=37, A/G=77, G/G=18. Two-sided p-values for indicated differences were obtained from DESeq2. FIG. 6B are box plots of mucus plug scores (2 measurements per subject), stratified into T2-low (n=41 subjects) and T2-high (n=71 subjects) groups based on sputum RNA-seq expression profiles. Two-sided p-values were obtained by fitting negative binomial mixed model implemented in SAS PROC GLIMMIX (METHOD=RSPL; DDFM=KENWARDROGER2) with subjectID as random effect. FIG. 6C are box plots of mucus plug scores (2 measurements per subject), stratified by both T2 status and ITLN1 rs4656959 variant genotype; T2-low subjects n: A/A=13, A/G=26, G/G=2 and T2-high subjects n: A/A=18, A/G=43, G/G=10. Two-sided p-values were obtained by fitting a negative binomial mixed model implemented in SAS PROC GLIMMIX (METHOD=RSPL; DDFM=KENWARDROGER2) with subjectID as random effect.

FIGS. 7A, 7B and 7C show IL-13 induction of ITLN-1 in mucociliary cultures derived from control and asthmatic airway brushes. FIG. 7A: Log normalized ITLN1 gene expression in BSA (control) and IL-13 treated ALI cultures generated from healthy (H; n=6) and asthmatic (A; n=13) participants. Boxplots, log fold change (LFC) values, and statistics are representative of data from donors with the ITLN1-expressing rs4656959 A/A or A/G genotype (H, n=3; A, n=13). Data points from donors with the ITLN1 rs4656959 variant G/G genotype described in this manuscript (H, n=3) are excluded from the statistical analyses and boxplots of gene expression between healthy and asthmatic-derived ALI cultures, but are included in these plots as green data points for transparency of all the data shown in FIG. 1b (n=19 total donors). FDR-adjusted two-sided p-value is based on a paired exact test (edgeR). FIG. 7B: normalized ITLN-1 peptides measured from independently collected apical aqueous ALI secretions following control and chronic IL-13 stimulation of mucociliary cultures generated from a subset of healthy (H, n=4) and asthmatic (A, n=10) donor cultures. Boxplots, log fold change (LFC) values, and statistics are representative of data from donors with the ITLN1-expressing rs4656959 A/A or A/G genotype (H, n=1; A, n=10). Data points from donors with the ITLN1 rs4656959 variant G/G genotype (H, n=3) are excluded from the statistical analyses and boxplots of protein detection between healthy and asthmatic-derived ALI cultures, but are included in these plots as green data points for transparency of all the data shown in FIG. 1f: Apical Washes-Aqueous Fraction (n=14 total donors). FDR-adjusted two-sided p-values are based on a paired exact test(edgeR). LFC=log fold change. FIG. 7C: normalized ITLN-1 peptides measured from independently collected apical ALI mucus fractions following control and chronic IL-13 stimulation of mucociliary cultures generated from healthy (H, n=4) and asthmatic (A, n=5). Boxplots, log fold change (LFC) values, and statistics are representative of data from donors with the ITLN1-expressing rs4656959 A/A or A/G genotype (H, n=2; A, n=4). Data points from donors with the ITLN1 rs4656959 G/G genotype (H, n=2; A, n=1) are excluded from the statistical analyses and boxplots of protein detection between healthy and asthmatic-derived ALI cultures, but are included in these plots as green data points for transparency of all the data shown in FIG. 1f: Apical Washes-Mucus Fraction (n=9 total donors). FDR-adjusted two-sided p-values are based on a paired exact test (edgeR). LFC=log fold change.

FIGS. 8A, 8B, 8C and 8D show IL-13 response markers of gene-edited mucociliary cultures. FIGS. 8A-8C: normalized MUC5AC, MUC5B, and SCGBIA1 expression measured by qPCR from tracheal mucociliary scrb- and ITLN1-edited ALI cultures following mock (BSA—black) or IL-13 (red) stimulation from 3 edited donors. Two-sided p-values calculated by Student's t-test based on comparisons between the calculated BSA:IL-13 fold-changes from each donor. FIG. 8D: representative dot blot images of apical washes collected from 3 gene-edited donors following mock or IL-13 stimulation (top), and quantified MUC5AC abundance from all edited donors (bottom). Two-sided p-values calculated using Student's t-test based on comparisons between BSA:IL13 changes in scrb and ITLN1 KO cultures, and based on BSA:IL13 fold changes for each donor between scrb and KO groups.

FIGS. 9A, 9B and 9C show ITLN-1 contributes to IL-13-mediated decrease in epithelial mucociliary function (mucociliary movement). FIG. 9A: box plots showing the log of average particle displacement within control edited (scrb) and ITLN1 KO mucociliary cultures that were mock—(BSA, white boxes) or IL-13-stimulated (grey boxes), with replicate experiments carried out using no wash, PBS wash, PBS-DTT wash, and ATP+PBS-DTT wash regimes. Average displacement values are colored by donor. There are 18 datapoints for each condition-donor pair (6 fields of view×3 inserts). Above plots are given the estimated % recovery (and associated p-values) of particle displacement in IL-13-stimulated cultures relative to BSA cultures when in an ITLN1 KO epithelium compared to the control (scrb) epithelium. Two-sided p-values are based on linear mixed model t-tests, with random effects for donor and insert, and use Satterthwaite approximation of degrees of freedom. FIGS. 9B and 9C: independently performed particle displacement experiment using the same 3 ITLN1 gene-edited tracheal donors re-cultured and differentiated at ALI for MCM assays. Box plots show the log of average particle speed (FIG. 9B) or average total displacement (FIG. 9C) within control edited (scrb) and ITLN1 KO mucociliary cultures that were mock—(BSA, white boxes) or IL-13-stimulated (grey boxes), with replicate experiments carried out using no wash, PBS wash, and PBS-DTT wash regimes. Average values are colored by donor. There are between 6 and 10 datapoints for each condition-donor pair (6-10 fields of view distributed across two different inserts per condition). Above the plots are given the estimated percent recovery (and associated p-values) of particle displacement (FIG. 9C) or speed (FIG. 9B) in IL-13-stimulated cultures relative to BSA cultures when in an ITLN1 KO epithelium compared to the control (scrb) epithelium. Two-sided p-values are based on linear mixed model t-tests, with a random effect for donor, and use Satterthwaite approximation of degrees of freedom.

FIGS. 10A, 10B and 10C show ITLN-1 contributes to IL-13-mediated decrease in epithelial mucociliary function (ciliary beat frequency). FIG. 10A: box plots of ciliary beat frequency (CBF) measured on control (scramble) and ITLN1 KO mucociliary ALI cultures from triplicate culture inserts and 3 donors following mock- and IL-13-stimulation. Each box plot represents all datapoints collected from a given insert and donor (white=BSA, grey=IL-13). Illustration of outliers was excluded for visualization purposes. P-values are based on linear mixed model t-tests with random effects for donor and insert and use Satterthwaite approximation of degrees of freedom. Two-sided p-values directly above boxes are for tests of differences in CBF between BSA and IL-13. P-values at the top of the panel are for tests of donor-specific differences in the CBF response to IL-13 between scramble and ITLN1 KO cultures. FIG. 10B: independently performed ciliary beat frequency (CBF) experiment using the same 2 ITLN1 gene-edited tracheal donors re-cultured and differentiated at ALI. Box plots of CBF are shown as measured on control (scramble) and ITLN1 KO mucociliary ALI cultures treated with mock- and IL-13-stimulation. There was one culture insert for each treatment for scramble and two inserts per treatment for ITLN1 KO. Each box plot represents all datapoints collected from all videos captured across donors and inserts for each condition (white=BSA, grey=IL-13). Two-sided p-values are based on linear mixed model t-tests with random effect for donor and use Satterthwaite approximation of degrees of freedom. FIG. 10C: box plots of CBF based on the validation experiment in b, where boxes are stratified by donor and insert. Two-sided p-values are based on mixed models implemented for scramble and ITLN1 KO separately; for the ITLN1 KO model, random effects were included for both donor and insert, whereas for the scramble model, a random effect was only included for donor.

FIGS. 11A, 11B and 11C show heparin binds to purified airway mucins and to the C-terminal domain of MUC5AC. FIG. 11A: bar graphs measuring binding of biotin-conjugated heparin to purified human airway mucins (isolated and pooled from n=5 subjects). Two-sided Student's T-test; *** p<0.001. FIG. 11B: bar graph measuring binding of biotin-conjugated heparin to the immobilized C-terminal domain peptides of MUC5AC. Two-sided Student's T-test; **** p<0.0001. FIG. 11C: bar graph measuring binding of biotin-conjugated heparin to the immobilized C-terminal domain peptides of MUC5B. Two-sided Student's T-test; ns=p-value not significant (p>0.05)

FIGS. 12A, 12B, 12C, 12D, 12E, 12F, 12G, 12H and 12I show mucus metaplastic marker relationships with ITLN1 expression and ITLN1 genotype. FIG. 12A: correlation plot of VST-normalized ITLN1 and MUC5AC expression in nasal brushings from the GALA II study (n=695). FIG. 12B: correlation plot of VST-normalized ITLN1 and MUC5B expression in nasal brushings from the GALA II study (n=695). FIG. 12C: box plots of log 2-normalized MUC5AC expression in nasal brushings from GALA II, stratified by T2 inflammation status and the ITLN1 variant rs4656959 genotype (T2-low n: A/A=141, A/G=148, G/G=35 and T2-high n: A/A=151, A/G=165, G/G=41). Two-sided p-values were obtained from DESeq2. FIG. 12 D: box plots of log 2-normalized MUC5B expression in nasal brushings from GALA II, stratified by T2 inflammation status and the ITLN1 variant rs4656959 genotype (T2-low n: A/A=141, A/G=148, G/G=35 and T2-high n: A/A=151, A/G=165, G/G=41). Two-sided p-values were obtained from DESeq2. FIG. 12E: box plots of the T2 mucus secretory network expression in the GALA II study stratified by T2 inflammation status and the ITLN1 variant rs4656959 genotype (T2-low n: A/A=141, A/G=148, G/G=35 and T2-high n: A/A=151, A/G=165, G/G=41). Two-sided p-values were obtained from DESeq2. FIG. 12F: correlation plot of VST-normalized ITLN1 and MUC5AC expression in sputum pellet samples from the SARP study (n=249). FIG. 12G: correlation plot of VST-normalized ITLN1 and MUC5B expression in sputum pellet samples from the SARP study (n=249). FIG. 12H: box plots of log 2-normalized MUC5AC expression in SARP sputum samples, stratified by T2 inflammation status and the ITLN1 variant rs4656959 genotype (T2-low n: A/A=43, A/G=61, G/G=13 and T2-high n: A/A=37, A/G=77, G/G=18). Two-sided p-values were obtained from DESeq2. FIG. 12I: box plots of log 2-normalized MUC5B expression in SARP sputum samples, stratified by T2 inflammation status and the ITLN1 variant rs4656959 genotype (T2-low n: A/A=43, A/G=61, G/G=13 and T2-high n: A/A=37, A/G=77, G/G=18). Two-sided p-values were obtained from DESeq2.

### DETAILED DESCRIPTION

The pathobiology underlying mucus obstruction of the airways and susceptibility to this phenotype remain some of the most enigmatic aspects of asthma and other obstructive lung diseases (Kesimer, M., et al. Airway Mucin Concentration as a Marker of Chronic Bronchitis. N Engl J Med 377, 911-922 (2017)). Most studies in this area have focused on the amount of mucus secreted (mucus hypersecretion), as well as distortions in both the concentration and ratio of MUC5AC and MUC5B mucin proteins in airway mucus (Lachowicz-Scroggins, M. E., et al. Abnormalities in MUC5AC and MUC5B Protein in Airway Mucus in Asthma. Am J Respir Crit Care Med 194, 1296-1299 (2016); Radicioni, G., et al. Airway mucin MUC5AC and MUC5B concentrations and the initiation and progression of chronic obstructive pulmonary disease: an analysis of the SPIROMICS cohort. Lancet Respir Med 9, 1241-1254 (2021)). However, the inventors recent finding that IL-13 drives formation of a distinct mucus secretory cell type, with a unique secretome, suggests that non-mucin proteins might contribute to mucus obstruction experienced by T2-high asthmatics (Jackson, N. D., et al. Single-Cell and Population Transcriptomics Reveal Pan-epithelial Remodeling in Type 2-High Asthma. Cell Rep 32, 107872 (2020)). As disclosed herein, ITLN1 is characterized, and was determined that it is induced in a switch-like fashion in the airway epithelium by IL-13 as part of a metaplastic mucus secretory network, and that ITLN-1 protein is incorporated into the airway mucus gel. Moreover, as disclosed herein, ITLN-1 can directly bind MUC5AC through electrostatic interactions and that its deletion partially reverses IL-13-induced mucostasis. Importantly, disclosed herein is a common eQTL variant for the ITLN1 gene. This variant drives a dramatic reduction in both gene and protein expression from the airway epithelium, which is associated with protection from mucus-plugging, specifically among T2-high asthmatics. The findings disclosed herein document one of the first non-mucin secreted airway proteins to be implicated in airway mucus obstruction in asthma. Targeting ITLN-1 in the airways, the loosen mucus viscosity in the airways provides one of the first non-mucin molecules that may be able to be used in patients exhibiting airway mucus plugging.

In contrast to the intestinal epithelium where ITLN1 is constitutively expressed by mucus secretory cells (Tsuji, S., et al. Human intelectin is a novel soluble lectin that recognizes galactofuranose in carbohydrate chains of bacterial cell wall. J Biol Chem 276, 23456-23463 (2001); Nonnecke, E. B., et al. Human intelectin-1 (ITLN1) genetic variation and intestinal expression. Sci Rep 11, 12889 (2021)), the inventors determined that there is little ITLN1 expression in the airway epithelium in the absence of T2 inflammation. In the airway, the inventors discovered that ITLN1 is expressed specifically within a gene network activated by a distinct population of IL-13-induced mucus secretory cells, explaining its switch-like induction in the airway of T2-inflamed individuals, particularly evident in the in vitro protein assays disclosed herein. The finding that airway epithelial ITLN1 expression is regulated by IL-13 confirms reports from several mouse and human studies (Gu, N., et al. Intelectin is required for IL-13-induced monocyte chemotactic protein-1 and -3 expression in lung epithelial cells and promotes allergic airway inflammation. Am J Physiol Lung Cell Mol Physiol 298, L290-296 (2010); Kuperman, D. A., et al. Dissecting asthma using focused transgenic modeling and functional genomics. J Allergy Clin Immunol 116, 305-311 (2005); Watanabe, T., et al. Expression of intelectin-1 in bronchial epithelial cells of asthma is correlated with T-helper 2 (Type-2) related parameters and its function. Allergy Asthma Clin Immunol 13, 35 (2017)). But the degree to which this IL-13 regulation occurs is striking—ITLN1 expression was induced by IL-13 more than 100-fold in primary bronchial and tracheal airway epithelial cells in culture and by 53-fold in brushed nasal airway epithelia from children with T2-high inflammation. Notably, 52% of variation in ITLN1 expression was explained by T2 status in children.

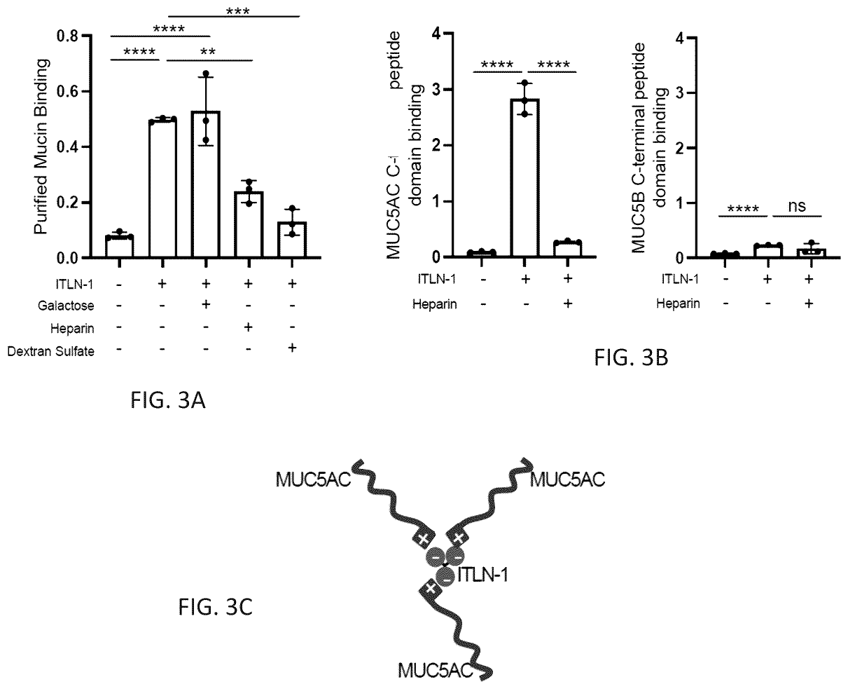

Deletion of ITLN-1 protein partially reverses the loss of mucociliary motion and ciliary beat frequency characteristics of IL-13-activated airway epithelial cells. This result led to considering whether ITLN-1 might directly bind mucin proteins to alter the biophysical properties of mucus. Similar to prior experiments (Kerr, S. C., et al. Intelectin-1 is a prominent protein constituent of pathologic mucus associated with eosinophilic airway inflammation in asthma. Am J Respir Crit Care Med 189, 1005-1007 (2014)), the inventors found that ITLN-1 did not bind human airway mucins in a galactose-dependent manner, consistent with prior findings that ITLN-1 selectively binds microbial glycan epitopes (Wesener, D. A., et al. Recognition of microbial glycans by human intelectin-1. Nat Struct Mol Biol 22, 603-610 (2015); Tsuji, S., et al. Human intelectin is a novel soluble lectin that recognizes galactofuranose in carbohydrate chains of bacterial cell wall. J Biol Chem 276, 23456-23463 (2001)). Instead, the inventors discovered that the negatively charged ITLN-1 binds the positively charged C-terminus of MUC5AC via electrostatic interactions. ITLN-1 did not bind the C-terminus of MUC5B even though, like MUC5AC, MUC5B has positively charged amino acids in its C-terminal domain (CTCK) (Xu, G., Forstner, G. G. & Forstner, J. F. Interaction of heparin with synthetic peptides corresponding to the C-terminal domain of intestinal mucins. Glycoconj J 13, 81-90 (1996)).—Given the homotrimeric nature of ITLN-1, it is possible that ITLN-1 could crosslink MUC5AC proteins via electrostatic interactions (FIG. 3C). Disulfide mucin cross-links increase the elastic behavior of mucus (Yuan, S., et al. Oxidation increases mucin polymer cross-links to stiffen airway mucus gels. Sci Transl Med 7, 276ra227 (2015)), and it is plausible that ITLN-1 could also cross-link mucin polymers to stiffen the airway mucus gel.

As disclosed herein, a naturally occurring correlate of the experimental ITLN-1 knock out (KO) in the form of a common human genetic variant that greatly diminishes ITLN1 airway gene expression is reported. Specifically, the inventors determined that induction of ITLN-1 by T2 inflammation is largely dependent on the rs4656959 genetic variant, constituting a striking gene-environment interaction that controls airway abundance of ITLN-1. Furthermore, in independent sets of primary bronchial and tracheal epithelial cultures, ITLN-1 protein is not produced or secreted by rs4656959 GG donor cultures, even in the presence of IL-13, suggesting that this variant functions as a protein knockout. Interestingly, the rs4656959 SNP is part of an 84-SNP linkage disequilibrium block (r2>0.8) in Europeans, which stretches up- and downstream of ITLN1 and has been associated with Crohn's disease in GWAS studies (Barrett, J. C., et al. Genome-wide association defines more than 30 distinct susceptibility loci for Crohn's disease. Nat Genet 40, 955-962 (2008); Franke, A., et al. Genome-wide meta-analysis increases to 71 the number of confirmed Crohn's disease susceptibility loci. Nat Genet 42, 1118-1125 (2010)). In stark contrast to the inventors findings in the airway epithelium, a study investigating the effects of this Crohn's disease risk haplotype on ITLN1 expression in the ileum and colon found that ITLN1 did not function as an eQTL in intestinal tissue (Nonnecke, E. B., et al. Human intelectin-1 (ITLN1) genetic variation and intestinal expression. Sci Rep 11, 12889 (2021)). This European haplotype also contains an ITLN-1 valine-to-aspartic acid coding variant (rs2274907), hypothesized to functionally mediate the Crohn's disease association. However, recent investigation of ITLN-1 protein isoforms derived from this variant, found no isoform-associated alterations to glycan binding kinetics, oligomerization, or protein stability (Nonnecke, E. B., et al. Human intelectin-1 (ITLN1) genetic variation and intestinal expression. Sci Rep 11, 12889 (2021)). Additionally, the inventors found only 19 variants are in strong LD with rs4656959 in our Puerto Rican population, of which rs2274907 is not one.

Occlusion of airways by mucus plugs has long been recognized as a cause of airway obstruction in acute severe asthma (including fatal cases of asthma). The inventors have quantified airway mucus plugging using a bronchopulmonary segment derived mucus plug score and found that high airway mucus plug scores are common in chronic severe asthma and are strongly associated with measures of type 2 inflammation and airflow obstruction in both cross-sectional and longitudinal studies (Dunican, E. M., et al. Mucus plugs in patients with asthma linked to eosinophilia and airflow obstruction. J Clin Invest 128, 997-1009 (2018); Tang, M., et al. Mucus Plugs Persist in Asthma, and Changes in Mucus Plugs Associate with Changes in Airflow over Time. Am J Respir Crit Care Med 205, 1036-1045 (2022)). Notably, using airway mucus plug scores in asthma patients genotyped for the ITLN1 eQTL variant, rs4656959, the inventors discovered that rs4656959 is associated with protection from mucus plugging, specifically among T2-high subjects. This finding suggests that ITLN-1 mediates pathologic mucus plug formation in T2-high asthma.

As demonstrated in the Examples herein, ITLN-1 contributes to T2-inflammation-induced airway mucostasis and airway plugging, and that a ITLN1 eQTL variant mediates risk of poor mucus-related outcomes in patients with severe T2-high asthma.

An “individual” is a vertebrate, such as a mammal, including without limitation a human. Mammals include, but are not limited to, farm animals, sport animals, pets, primates, mice and rats. The term “individual” can be used interchangeably with the term “animal”, “subject” or “patient”.

As used herein, reference to a reference or control, means a subject who is a relevant reference or control to the subject being evaluated by the methods of the present invention. The control can be matched in one or more characteristics to the subject. In one aspect, the control can be an individual with no history of an airway inflammatory disease such as a T2-high airway inflammatory disease, such as T2-high asthma. In one aspect, the control does not have T2-high asthma. In another aspect, the control has been determined to be asthma-free. The control can be matched in one or more of the following characteristics, gender and age. The reference or control protein and/or RNA expression level used in the comparison of the methods of the present invention can be determined from one or more relevant reference or control subjects.

In other various aspects of the invention, the subject can be treated for an inflammatory disease of the airways, such as for a T2-high inflammatory disease of the airway, including but not limited to T2-high asthma, by various methods including but not limited to, administration of a T2 pathway inhibitor drug, a bronchodilator, an inhaled corticosteroid, administration of a phosphodiesterase inhibitor, administration of an antibiotic, administration of prednisone, antibiotics, pulmonary rehabilitation, oxygen therapy, and combinations thereof, as well as, by known standard of care methods for the diseases.

The term “sample” or “patient sample” or “subject sample” or “test sample” can be used generally to refer to a sample of any type which contains products that are to be evaluated by the present methods, including but not limited to, a blood sample, a sample of isolated cells, a tissue sample and/or a bodily fluid sample. A biological sample can include any bodily fluid or tissue from a subject that may contain the proteins contemplated herein, as well as the RNA and genes that encode the proteins. In some embodiments, the sample may comprise blood, plasma or peripheral blood mononuclear cells (PBMCs), leukocytes, monocytes, lymphocytes, basophils or eosinophils. In a preferred aspect, the biological sample is blood. In one aspect, the methods of the present invention can be performed on an ex vivo biological sample.

As used herein, the term “expression”, when used in connection with detecting the expression of a gene, can refer to detecting transcription of the gene (i.e., detecting mRNA levels) and/or to detecting translation of the gene (detecting the protein produced). To detect expression of a gene refers to the act of actively determining whether a gene is expressed or not. This can include determining whether the gene expression is upregulated (or increased) as compared to a control, downregulated as compared to a control, or unchanged as compared to a control or increased or decreased as compared to a reference or control level. Therefore, the step of detecting or determining expression does not require that expression of the gene actually is upregulated or downregulated or increased or decreased, but rather, can also include detecting or determining that the expression of the gene has not changed (i.e., detecting no expression of the gene or no change in expression of the gene).

Expression of transcripts and/or proteins is measured by any of a variety of known methods in the art. For RNA expression, methods include but are not limited to: extraction of cellular mRNA and Northern blotting using labeled probes that hybridize to transcripts encoding all or part of the gene; amplification of mRNA using gene-specific primers, polymerase chain reaction (PCR), and reverse transcriptase-polymerase chain reaction (RT-PCR), quantitative PCR, and/or RNA Ampliseq, followed by quantitative detection of the product by any of a variety of means; multiplexed quantitative PCR enrichment of cDNA amplicons, followed by conversion of amplicons to sequence libraries and Next-generation based sequencing of libraries to generate digital count expression data; extraction of total RNA from the cells, which is then labeled and used to probe cDNAs or oligonucleotides encoding the gene on any of a variety of surfaces; in situ hybridization; and detection of a reporter gene.

Methods to measure protein expression levels generally include, but are not limited to: mass spectrometry, Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry, as well as assays based on a property of the protein including but not limited to enzymatic activity or interaction with other protein partners. Binding assays are also well known in the art. For example, a BIAcore machine can be used to determine the binding constant of a complex between two proteins. The dissociation constant for the complex can be determined by monitoring changes in the refractive index with respect to time as buffer is passed over the chip (O'Shannessy et al., 1993, Anal. Biochem. 212:457; Schuster et al., 1993, Nature 365:343). Other suitable assays for measuring the binding of one protein to another include, for example, immunoassays such as enzyme linked immunoabsorbent assays (ELISA) and radioimmunoassays (RIA); or determination of binding by monitoring the change in the spectroscopic or optical properties of the proteins through fluorescence, UV absorption, circular dichroism, or nuclear magnetic resonance (NMR).

The following examples are provided for illustrative purposes, and are not intended to limit the scope of the invention as claimed herein. Any variations which occur to the skilled artisan are intended to fall within the scope of the present invention.

### EXAMPLES

### Example 1: Materials and Methods for the Examples Described Herein

### Human Subject Information

All research conducted in these studies complies with all relevant ethical regulations. Human bronchial and tracheal airway epithelial cells from adult subjects were banked in a de-identified fashion and obtained for research purposes from the National Jewish Health (NJH) Live Cell Core. The NJH Live Cell Core is an institutional review board-approved study (HS-2240) for the collection of tissues from consented patients for researchers at NJH. For tracheal epithelial cell isolation, human lung specimens were obtained from de-identified lung donors whose lungs were not suitable for transplantation from the International Institute for the Advancement of Medicine (Edison, NJ), and Donor Alliance of Colorado. Written consent for research on tracheal and lung specimens was obtained by the Organ Procurement Organization prior to organ donation. The National Jewish Health Institutional Review Board (IRB) approved the research on lung cells under IRB protocol HS-3209. Nasal airway epithelial brushes were collected from subjects recruited as part of the Genes-environments and Admixture in Latino Americans II (GALA) childhood asthma study to be used in genome-wide genetic and genomic analysis, which was approved by local institutional review boards (UCSF, IRB number 10-00889, Reference number 153543, NJH HS-2627). All subjects and their parents provided written informed assent and written informed consent for sample collection and use of the samples in genetic and genomic analyses (Neophytou, A. M., et al. Air Pollution and Lung Function in Minority Youth with Asthma in the GALA II (Genes-Environments and Admixture in Latino Americans) and SAGE II (Study of African Americans, Asthma, Genes, and Environments) Studies. Am J Respir Crit Care Med 193, 1271-1280 (2016); Nishimura, K. K., et al. Early-life air pollution and asthma risk in minority children. The GALA II and SAGE II studies. Am J Respir Crit Care Med 188, 309-318 (2013)). Bronchial airway brushings used for scRNAseq were collected following informed consent and assent as part of the Epithelial Barriers in Asthma, Eczema, Food Allergy, and GERD study approved by the BRANY IRB for National Jewish Health under protocol HS-3255-528. Sputum specimens and mucus plug scores were collected following informed consent from participants, into the Severe Asthma Research Program (SARP 3) study, and the study was approved by the IRBs at all participating centers.

### ALI Culture and IL-13 Stimulation of Primary Bronchial and Tracheal Airway Epithelial Cells

For both primary human bronchial (n=19) and tracheal (n=5 per ITLN1 genotype group) epithelial samples, basal cells were cultured for expansion using a modified Schlegel method as previously described (Reynolds, S. D., et al. Airway Progenitor Clone Formation Is Enhanced by Y-27632-Dependent Changes in the Transcriptome. Am J Respir Cell Mol Biol 55, 323-336 (2016); Everman, J. L., Rios, C. & Seibold, M. A. Utilization of Air-Liquid Interface Cultures as an In Vitro Model to Assess Primary Airway Epithelial Cell Responses to the Type 2 Cytokine Interleukin-13. Methods Mol Biol 1799, 419-432 (2018)). Primary basal bronchial cells were seeded onto 6.5 mm, 0.4 um pore transmembrane inserts pre-coated with PureCol bovine collagen (2×10⁴ cells/insert) in ALI expansion medium supplemented with Y-27632. Primary tracheal cells were seeded onto 6.5 mm 24-well polyester Transwell inserts with 0.4 μm pore size (4×10⁴ cells/insert) in PneumaCult Expansion Plus medium (StemCell Technologies) supplemented with Y-27632. Cultures were air-lifted upon development of an intact monolayer and basolateral media was changed to PneumaCult ALI (PC-ALI) media to allow for epithelial differentiation. At day 11 post-airlift paired cultures triplicate inserts (n=3) per treatment condition were stimulated with either IL-13 (10 ng/ml) or BSA supplemented media (mock control) daily for 10 days and harvested for experiments as described below.

### Immunofluorescence Labeling and Confocal Imaging

Following mock or IL-13 stimulation, ALI cultures were fixed in 3.2% paraformaldehyde/PBS prior to labeling. For histological section labeling, fixed ALI inserts from 8 paired donor cultures were paraffin embedded and sectioned. Histology sections were stripped of paraffin using HistoChoice Clearing Agent (SIGMA®), rehydrated using a decreasing gradient of alcohol washes (100%, 90% 70%, 50%, 30%, 0%), and antigen retrieval conducted using Citric Acid-based Antigen Unmasking Solution pH 6.0 (Vector Laboratories). Prior to labeling, all samples were blocked and permeabilized for 30 minutes using 3% BSA/0.1% Triton X-100 in tris-buffered saline (TBS). Primary labeling was conducted using 3% BSA/0.1% Triton X-100 in TBS for 1 hour with antibodies against MUC5AC (1:500; FISHER® SCIENTIFIC), MUC5B (1:200; SantaCruz sc-20119), or ITLN-1 (1:200; AbCam ab118232). Samples were washed twice using TBS/0.1% Triton X-100 (TBST) and secondary labeling was conducted using DAPI (1:1000), AlexaFluor488 goat anti-rabbit IgG and AlexaFluor594 goat anti-mouse IgG (1:500; ThermoScientific) for 30 minutes. Slides were washed twice in TBST and mounted with Vectashield HardSet Antifade Mounting Media (Vector Laboratories). Fluorescent images of histological sections were acquired using a Revolve R4 microscope (Echo Labs). Whole mount ALI sections from 3 donors were fixed, blocked, and labeled as described above and Z-stack images were captured using a Zeiss LSM700 Confocal microscope. Confocal image analysis and annotation was performed using Zeiss ZEN Blue Lite analysis software.

### Proteomic Sample Preparation and Mass Spectrometry

For aqueous fraction proteomic analyses, apical washes from paired BSA (mock) and IL-13-treated ALI cultures were collected from inserts cultured in triplicate from a subset of donors from the HBEC experiments (n=14) individually in Fisher 2 ml low binding microfuge tubes and samples were centrifuged at 225×g at 4° C. to remove intact cells from the washes. Supernatants were collected from each sample tube and the visible insoluble (i.e., mucus) fraction was removed from the soluble (aqueous) fraction. The soluble apical fraction was mixed with 3× volume of ice cold LC/MS grade methanol followed by incubation at −80° C. for 1 hour. Proteins were then pelleted by centrifugation at 18,000×g and the supernatant was removed. Protein pellets were dried in a SpeedVac at 45° C. and frozen at −80° C. For trypsin digestion, protein pellets from 2 of the harvested culture inserts per condition were processed and pooled for analysis as described below. Samples were denatured and reduced using 50% trifluoroethanol in 50 mM ammonium bicarbonate with 0.1 mM DTT at 65° C. for 45 minutes. Proteins were then alkylated for 1 hour in the dark using 0.32 mM iodoacetamide (IAA), and excess IAA was then reduced using 0.08 mM DTT for 1 hour in the dark. Samples were diluted with 400 ul of 25 mM ammonium bicarbonate buffer and digested with 0.3 ug of trypsin for 5 hours at 37° C. Digests were then stopped using 2 μl of formic acid and duplicate samples were combined and dried at 45° C. in a SpeedVac concentrator and dried samples were stored at −70° C. For mass spectrometry, the soluble fraction samples were first resuspended with 10 ul of 3% Acetonitrile+0.1% formic acid and the total sample was analyzed using a nanoAdvanced UPLC (Bruker) with a 15 cm×100 um ProntoSil C18AQ column and 2 cm trap column (nanoLCMS Solutions). Mobile phase was H₂O+0.1% FA (A) and acetonitrile+0.1% FA (B), peptides were separated using a gradient of 2-40% B over 30 minutes at a flow rate of 800 nL/minute and with the column temperature kept constant at 40° C. The column was connected through a Captive Spray nano source to an Impact Q-TOF (Bruker). Data were collected over a mass range of 150-2200 m/z using a 1 Hz MS scan and a total duty cycle time of 2 seconds. Data were processed using DataAnalysis 4.2 (Bruker), compounds were searched against the SwissProt database using Mascot 2.4 (Matrix Science) with the percolator algorithm, and protein and peptide results were assessed and filtered with ProteinScape 3.0 (Bruker). Raw peptide counts were normalized to the total number of peptides measured within each sample, and differential presence was analyzed by EdgeR analysis using FDR<0.05 as a significance cut-off.

For mucus fraction proteomics, apical washes from paired BSA (mock) and IL-13-treated ALI cultures were collected from inserts cultured in triplicate from a subset of donors used in the HBEC experiments (n=9) individually in Fisher 2 ml low binding microfuge tubes and samples were centrifuged at 225×g at 4° C. to remove intact cells from the washes. Supernatants were collected from each sample tube and the visible insoluble (i.e., mucus) fraction was recovered, dried in a SpeedVac at 45° C., and stored at −80° C. Frozen mucus fraction pellets from 2 of the harvested culture inserts per condition were processed as detailed below and pooled for analysis. Samples were processed and digested following the Preomics Sample Preparation Kit for Pelleted Cells and Precipitated Proteins v2.5. Briefly, mucus pellets were suspended in 25 μl of Lyse buffer, vortexed for 5 min, then incubated at 95° C. for 10 min and vortex mixed prior to pooling and sonication for 10 cycles of 30 sec On/Off. Digest buffer was diluted 1:4 with Resuspend buffer, and then mixed 1:1 with sample in Lyse buffer prior to digestion for 3 hrs and 10 min at 37° C. Sample digestion was stopped, washed twice, and eluted. Final eluates were dried in a speed vac at 45° C. and stored overnight at −20° C. Samples were suspended in Preomics Load buffer and bath sonicated for 10 cycles of 30 sec On/Off and stored at −70 C. Samples were analyzed using LCMS methods similar to previous publication (Yu, Q., et al. Benchmarking the Orbitrap Tribrid Eclipse for Next Generation Multiplexed Proteomics. Anal Chem 92, 6478-6485 (2020)). Samples were loaded onto a 2 cm PepMap 100, nanoviper trapping column and chromatographically resolved on-line using a 0.075×150 mm, 3.0 μm EASY-Spray PepMap RSLC C18 reverse phase nano column (Thermo Scientific) using a Ultimate 3000 RSCLnano LC system (Thermo Scientific). Mobile phases consisted of water+0.1% formic acid (A) and 100% acetonitrile+0.1% formic acid (B). Samples were loaded onto the trapping column at 5.0 μL/min for 4 minutes at initial condition before being chromatographically separated at a flow rate of 300 nl/min using a gradient of 3-7% B over 3 minutes, 7-22% B over 37 minutes, and 22-33% B over 5 minutes for a total 45 minute gradient at 40° C. The gradient method was followed by a column wash at 70% B for 5 minutes. Data was collected on an Orbitrap Eclipse (Thermo Scientific) operated using intensity dependent CID MS/MS to generate peptide ID's. MS1 spectra were acquired in the Orbitrap (Resolution=120 k; AGQ target=100%; MaxIT=Auto; RF Lens=30%; mass range=350-1500; Profile data). Precursors selected for MS/MS were filtered by MIPS model set to peptide with an intensity threshold of 15000 and only charge states 2-6 were allowed. Dynamic exclusion was employed for 14 s excluding all charge states for a given precursor. MS2 spectra were collected using CID in the linear ion trap (Isolation window=1 m/z [quadrupole], rate=rapid; AGQ target=Standard; MaxIT=Dynamic; CID=35%). Data were searched and extracted using SEQUEST HT and the label-free quantitation workflow in Proteome Discover software version 2.5.0.400 utilizing the minora feature detector, feature mapper and precursor ions quantifier algorithms. Spectra were searched against the SwissProt Homo sapiens database allowing up to 2 missed tryptic cleavages with fixed carbamidomethyl (C) and dynamic deamidated (N,Q) and oxidation (M) modifications. The monoisotopic peptide mass tolerance allowed was +6.0 ppm and the MS/MS tolerance was ±0.4 da. Peptides were adjusted to a 1% false discovery rate (FDR) using the percolator algorithm. Additionally, only high confidence peptides were allowed for protein scoring and a protein FDR cut-off of 1% was also used. A signal to noise (S/N) threshold of 3 was set for minora feature detector and peptide spectrum matches (PSMs) confidence levels were set to high for feature ID linking. A coarse retention time (RT) alignment of data was performed using max RT shift of 10 min and mass error of 6 ppm. Following RT alignment a RT tolerance of 1.4 min, mass tolerance of 6 ppm, minimum S/N threshold of 5 were set for feature linking and mapping. Precursor quantification was based on ion intensity. Raw peptide level abundance and identification data was exported from proteome discover and filtered to peptides found in 100% of samples in 1 of 6 conditions. Peptide level data was then rolled up into protein level data by summing the abundance of all unique peptides for a specific protein. Raw peptide counts were normalized to the total number of peptides measured within each sample, and differential presence was analyzed by EdgeR analysis using FDR<0.05 as a significance cut-off.

### Lentiviral CRISPR-Cas9 Gene Editing of Tracheal Airway Epithelial Cells

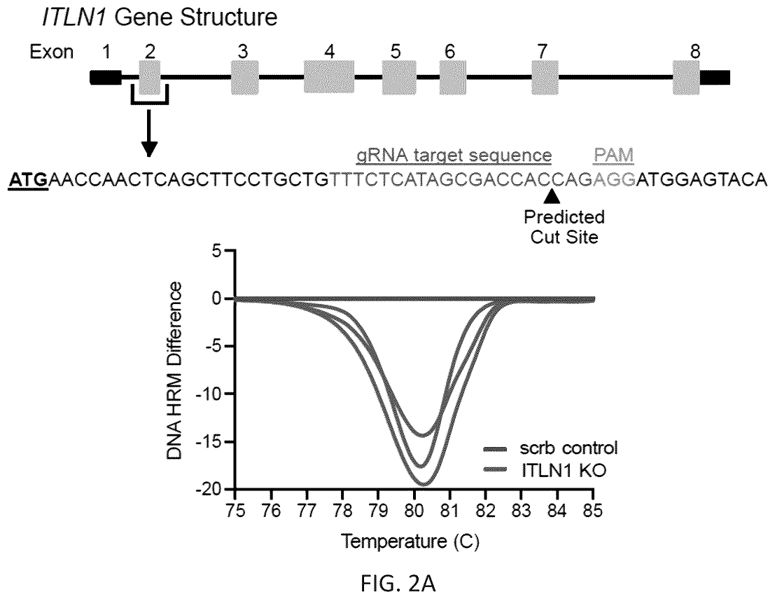

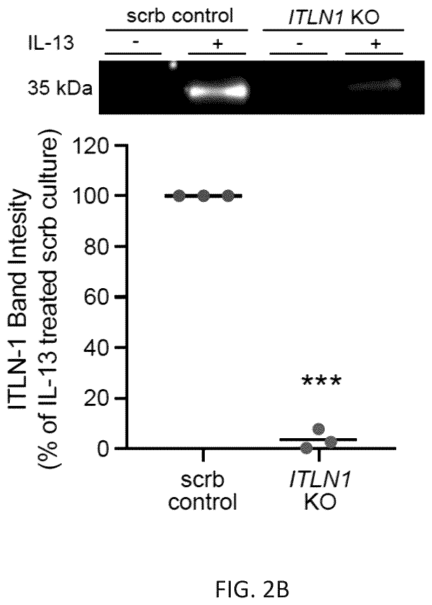

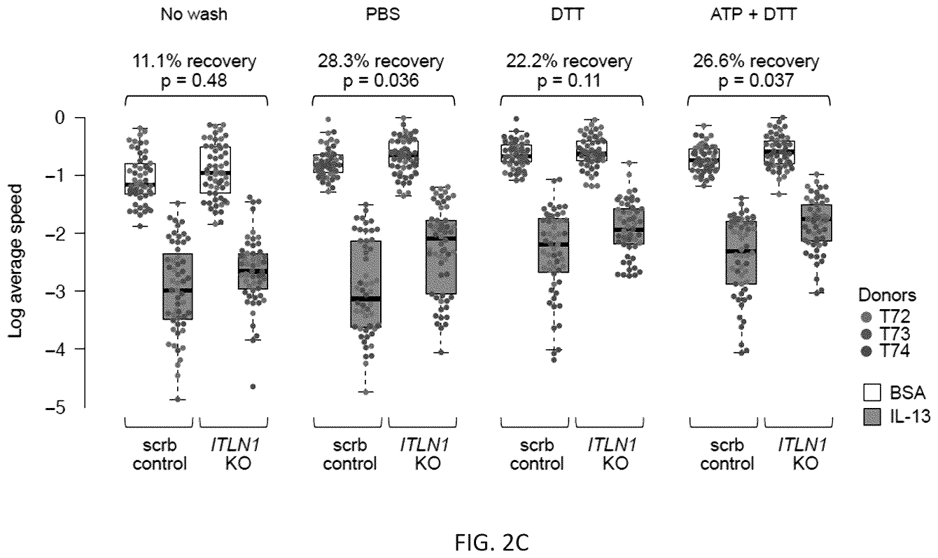

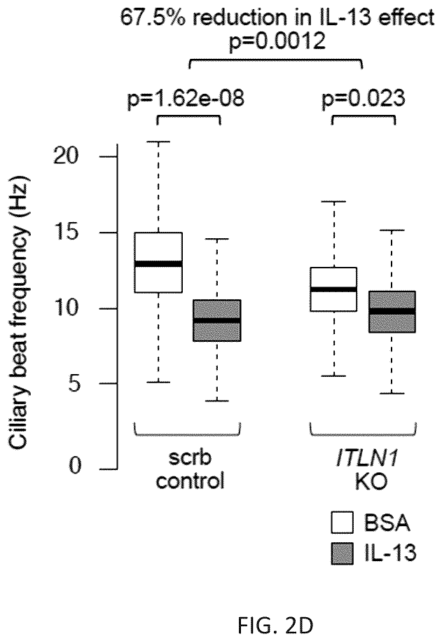

The design of the CRISPR targeting guide sequences, addition of adaptors and cloning into the lentiCRISPR plasmid backbone, propagation and titration of lentivirus, and AEC transduction and selection was performed as previously described (Chu, H. W., et al. CRISPR-Cas9-mediated gene knockout in primary human airway epithelial cells reveals a proinflammatory role for MUC18. Gene Ther 22, 822-829 (2015); Everman, J. L., Rios, C. & Seibold, M. A. Primary Airway Epithelial Cell Gene Editing Using CRISPR-Cas9. Methods Mol Biol 1706, 267-292 (2018)). The gRNA sequences were designed using the CRISPRdesign (version 1) tool offered by the Zhang lab at MIT (crispr.mit.edu) and chosen to target exon 2 of the ITLN1 gene (Forward guide: ITLN1-75ex2gRNAFwd 5′-CACCGTTTCTCATAGCGACCACCAG-3′(SEQ ID NO:1); Reverse guide: ITLN1-75ex2gRNARev 5′-AAACCTGGTGGTCGCTATGAGAAAC-3′(SEQ ID NO:2); FIG. 2A). A scrambled sequence was designed and used as a control in parallel for all CRISPR gene editing experiments (forward scramble guide 5′-CACCGCGTGCTCCGTTCGCGCTTC-3′(SEQ ID NO:3); reverse scramble guide 5′-AAACGAAGCGCGAACGGAGCACGC-3′ (SEQ ID NO:4)). Screening primers were designed over the CRISPR-Cas9-targeted region of the ITLN1 gene for high resolution melt curve analysis using MeltDoctor HRM Master Mix on a QuantStudio 6 Flex Real-Time PCR System (Life Technologies). Inference of CRISPR Edits (ICE) analysis was performed using the ICE Analysis tool provided by Synthego (Conant, D., et al. Inference of CRISPR Edits from Sanger Trace Data. CRISPR J 5, 123-130 (2022)). DNA was isolated from control scrb- and ITLN1-targeted expanded basal cells prior to being seeded to transwell inserts. PCR amplification was performed using 25 ng of input DNA from each sample, with 0.2 μM of ITLN1_ICE1_Fwd primer (5′-GGCTGGAAGGTGACACAGTT-3′) (SEQ ID NO:5) and 0.2 uM ITLN1_ICE1_Rev (5′-CCCAAAACCAACACCAACTC-3′) (SEQ ID NO:6) primers using CloneAmp HiFi PCR PreMix (Takara Bio) as per manufacturer's instructions. PCR amplicons were purified using the QIAquick PCR Purification Kit (Qiagen), and submitted for Sanger sequencing using the ITLN1_ICE1_SeqF primer (5′-GACACAGTTCTTGCCACAGC-3′) (SEQ ID NO:7). Early passage primary tracheal airway epithelial cells were used for transduction in gene-editing experiments (n=3 donors). Following antibiotic selection, gene edited basal epithelial cells were seeded onto 6.5 mm 24-well polyester Transwell inserts with 0.4 μm pores at 3.0×10⁵ cells/cm² in ALI Expansion Media supplemented with Y-27632, and were air-lifted upon development of an intact monolayer. Cultures were differentiated using PneumaCult ALI (PC-ALI) differentiation media (StemCell Technologies) and IL-13 stimulations were performed as described above.

### SDS-PAGE/Western Blot and Dot Blot Analysis

ALI cultures were mock or IL-13 stimulated in triplicate, as described above, and warm PBS was added to the apical chamber of each culture for 10 min at 37° C. prior to harvest and pooling. For SDS-PAGE apical wash samples were denatured at 94° C. for 7 min in Laemmli buffer and loaded onto a BioRad TGX 4-20% SDS-PAGE gel at 150V for 1 hour and transferred to nitrocellulose at 90V for 2 hours. For dot blots, 2 ul, of each apical wash samples was applied to a nitrocellulose membrane and allowed to air dry. For all Western blots, membranes were blocked with 4% milk/0.1% Tween-20/TBS buffer and immunoblots were conducted using either rabbit anti-ITLN-1 (1:200; AbCam ab118232) in 4% milk/0.1% Tween-20/TBS overnight at 4° C., or mouse anti-MUC5AC (1:1000; Fisher Scientific MA1-21907) in 4% milk/TBS/0.1% Tween-20 for 1 hour at 4° C. Blots were probed with goat anti-rabbit (BioRad #170-6515) or goat anti-mouse (1:10,000; BioRad #170-6516HRP-conjugated antibodies for 1 hour and incubated with ClarityMax Western ECL Blotting Substrate for chemiluminescent detection of protein bands. Images were captured on a BioRad ChemiDoc and band intensity densitometry was measured using ImageJ.

### Mucociliary Movement Analysis Using Particle Tracking

Control (scrb) and ITLN1 KO ALI cultures described above were treated with BSA or IL-13 prior to particle tracking assays. Each insert was placed onto a plastic petri dish, and 20 ul of Cy5-labeled 5 um beads (Bangs Laboratory, Inc; 1:500 beads diluted in ALI culture media) were added to the apical chamber of each culture using a P20 pipet. For each sample, 10 sec videos were captured on 4× magnification over 6 fields of view per culture insert, using triplicate culture inserts per sample treatment (BSA and IL-13) from all 3 edited donors (n=18 total videos per donor-treatment pair; 3 inserts×6 videos). Imaging was repeated for these same inserts after each of the following wash conditions: (1) No Wash, to capture the native MCM after 10d IL13 stimulations, (2) PBS wash (10 min at 37° C.), aimed at hydrating and removing soluble mucus material, (3) PBS-DTT wash (10 mM DTT in PBS for 10 min at 37° C.) aimed at hydrating and denaturing the mucus layer, and (4) ATP+PBS-DTT wash (100 uM ATP in PBS in apical side of ALI cultures for 1 hour at 37° C. followed by 10 mM DTT wash) aimed at inducing secretion of intracellular mucus from mock and IL-13 stimulated cultures, followed by PBS-DTT wash aimed at hydration and denaturation of the ATP-induced mucus secretions. MOV-formatted videos were converted to greyscale and then imported into the Trackpy Python package for particle tracking analysis (Allan D. B.; Caswell, T. K. N. C.v.d. W., C. M.; and Verweij, R. W. soft-matter/trackpy: Trackpy v0.5.0 (v0.5.0). (2021)). Briefly, particles were detected within frames (trackpy.batch function with diameter=21 and minmass=10) and then linked across frames into movement trajectories (tp.link_df function with search_range=10 and memory=10), where we removed particles not present in at least 25% of frames. For each video, based on an average of 305 detectable particles per video, we calculated the geometric mean of both total Euclidean displacement of each particle and total particle speed, as calculated by total particle displacement divided by the number of frames between first and last observance of a particle. We then estimated and tested the extent that the log of mean particle displacement or speed differed based on treatment (BSA control versus IL-13), ITLN1 KO status (mock-targeted versus ITLN1 KO), or their interaction for each wash regime (no wash, PBS wash, PBS-DTT wash, ATP+PBS-DTT wash) using a mixed model, where we included a random intercept for each donor and insert. Mixed models were run using the lmerTest R package (Kuznetsova, A., Brockhoff, P. B. & Christensen, R. H. B. lmerTest Package: Tests in Linear Mixed Effects Models. Journal of Statistical Software 82, 1-26 (2017)) and p-values were calculated based on Satterthwaite-approximated degrees of freedom. The independently replicated particle tracking experiment was performed on the same 3 gene edited donors as used above, following re-differentiation at ALI, using a slightly different design as described below. For each sample, 10 sec videos were captured on 4× magnification over 4 fields of view per culture insert, using duplicate culture inserts per sample treatment (BSA and IL-13) from all 3 edited donors (n=average of 8 total videos per donor-treatment pair). Imaging was repeated for these same inserts after each of the following wash conditions: (1) No Wash, to capture the native MCM after 10d IL13 stimulations, (2) PBS wash (10 min at 37° C.), aimed at hydrating and removing soluble mucus material, and (3) PBS-DTT wash (10 mM DTT in PBS for 10 min at 37° C.) aimed at hydrating and denaturing the mucus layer. Quantification of total displacement, particle speed, and all associated analyses were performed similar to that described above.

### Measurement of Ciliary Beat Frequency

Using tracheal ALI cultures from control or ITLN1-targeted CRISPR-Cas9 gene editing experiments performed on 3 donors, triplicate ALI culture inserts were stimulated with BSA or IL-13 as described above, and were used to measure ciliary beat frequency (CBF) to assess functional response to IL-13 in these epithelia. ALI cultures were first washed once in 37° C. PBS for 10 min, after which the wash was removed and cultures were imaged by placing the plastic insert in a glass-bottom Petri dish with PC-ALI medium. Digital high-speed videos were recorded on 15 different fields of view across the outer perimeter edge of the sample, (FOV=1280×1024 px, 1 px=0.1625 m), under bright-field illumination with DIC optics and at a sampling frequency of 170 fps using a Nikon Eclipse Ti-E inverted microscope (Nikon Instruments, Japan) with a 40×objective (Plan Apo X 40×, N. A. 0.75, Nikon) fitted with an ANDOR ZYLA® sCMOS camera. Epithelial cultures were imaged using a LIVECELL™ stage top incubator, chamber, where temperature, CO₂ and humidity were continuously monitored and maintained at values of 37° C., 5%, and 90%. Any videos that showed drifting or duplicated areas of analysis were not included in the analyses. CBF was quantified using Differential Dynamic Microscopy, as previously described (Chioccioli, M., et al. Quantitative High-Speed Video Profiling Discriminates between DNAH11 and HYDIN Variants of Primary Ciliary Dyskinesia. Am J Respir Crit Care Med 199, 1436-1438 (2019); Feriani, L., et al. Assessing the Collective Dynamics of Motile Cilia in Cultures of Human Airway Cells by Multiscale DDM. Biophys J 113, 109-119 (2017)). Briefly, each field of view was first divided into tiles of 64×64 px (˜10 um per side). The distribution of CBF in the field of view was then measured by running DDM on the tiles that showed motion and fitting its output with an empirical oscillating function (the Image Structure Function). The CBF distributions for each treatment were then built by pooling the values of CBF (in units of Hz) measured on the “active” tiles of all the fields of view imaged across the samples. Total data points are listed below and were collected across triplicate culture inserts per treatment, editing condition, and donor: scrb control:BSA=30,899; scrb control:IL-13=24,786, ITLN1 KO:BSA=25,533, ITLN1 KO:IL-13=24,964. We then estimated and tested the extent that CBF differed based on treatment (BSA control versus IL-13), ITLN1 KO status (mock-targeted versus ITLN1 KO), or their interaction using a mixed model, where we included a random intercept for each donor and insert. Mixed models were run using the lmerTest R package and p-values were calculated based on Satterthwaite-approximated degrees of freedom. Video capture for CBF analysis in the validation experiment differed slightly, as described below. Data was captured from 2 of the cultured and differentiated donors, as used in the previous experiment, and underwent either BSA (mock) or IL-13 stimulation. Videos were collected from 1 insert from each stimulation condition per donor for the scrb controls, and 2 inserts from each stimulation condition per donor for the ITLN1 KO samples. The digital high-speed videos were recorded on 5 different fields of view across the sample (FOV=1920×1024 px, 1 px=0.146 μm), under bright-field illumination and at a sampling frequency of 150 fps using a Nikon Eclipse Ti-E inverted microscope (Nikon Instruments, Japan) with a 40× objective (Plan Apo X 40×, N. A. 0.95, Nikon) fitted with a GRASSHOPPER®3 GS3-U3-23S6M-C CMOS camera (FLIR Integrated Imaging Solutions GmbH, Germany). Epithelial cultures were imaged in a custom-made chamber where temperature, CO₂ and humidity were continuously monitored and maintained at values of 37° C., 5%, and 90%, respectively. CBF was quantified and analyzed in a similar manner as described above resulting in total data points listed here: scrb control:BSA=3,061; scrb control:IL-13=1,640, ITLN1 KO:BSA=5,519, ITLN1 KO:IL-13=4,887.

### Quantitative PCR Gene Expression Analysis

Experiments using tracheal airway ALI epithelial cultures to validate the gene expression effects of ITLN1 rs4656959 (n=5 subjects per genotype), and from tracheal CRISPR gene editing experiments were harvested in RNA/DNA Lysis Buffer supplemented with 40 mM dithiothreitol (DTT). Triplicate culture inserts per condition were pooled and lysates were extracted using the Zymo MiniRNA Kit (Zymo Research) and cDNA was synthesized using the Maxima First Strand cDNA Synthesis Kit for qRT-PCR (Thermo Scientific). For qPCR analysis, cDNA samples were amplified and analyzed using a QuantStudio 6 Flex RealTime PCR System (Life Technologies). Assays were run using Brilliant III Master Mix (Agilent) using the following cycling conditions: 95° C. for 3 minutes, followed by 40 cycles of 95° C. 10 seconds and 60° C. for 30 seconds using PrimeTime qPCR 5′ Nuclease probe assays for GusB (Hs.PT.51.2648420; IDT), ITLN1 (Hs.PT.58.39839277; IDT), MUC5AC (Hs01365600_g1; Thermo), MUC5B (Hs.PT.51.5074152.gs; IDT), and SCGB1A1 (HS.PT.58.1190800; IDT). Expression of was normalized to the airway epithelial housekeeping gene (GUSB).

### Human Mucin and Peptide Binding Assays

Human purified mucins were obtained from asthmatic induced sputum by purification of high molecular weight proteins, as described previously (Kerr, S. C., et al. Intelectin-1 is a prominent protein constituent of pathologic mucus associated with eosinophilic airway inflammation in asthma. Am J Respir Crit Care Med 189, 1005-1007 (2014); Royle, L., et al. Glycan structures of ocular surface mucins in man, rabbit and dog display species differences. Glycoconj J 25, 763-773 (2008)). Five asthmatic donors were used to collect and purify mucins, and samples were pooled together for the mucin binding assays. Human purified mucins, MUC5AC C-terminal recombinant peptide (AA5568-AA5654, Mybiosource, #MBS2009104) or MUC5B C-terminal recombinant peptide (AA5653-AA5762, LifeSpan Biosciences, G12612) were coated on a NUNC Maxisorp plate overnight at 4° C. at the concentration of 20 pg/mL in carbonate-bicarbonate buffer, pH=9.6. Plates were then blocked with blocking buffer (TBS, CaCl₃) 3 mM, BSA 3%, Tween-20 0.05%) for 2 hours at room temperature. FLAG-tagged recombinant ITLN-1 (Sigma, SRP8047) was incubated for 2 hours at 10 pg/mL in binding buffer B (TBS, 3 mM CaCl₃), 1% BSA, 0.05% Tween-20, 50 mM EDTA) for purified mucin binding or binding buffer A (TBS, 3 mM CaCl₃), 1% BSA, 0.05% Tween-20) for recombinant peptide binding. For binding inhibition assays, heparin (150 pg/mL, Sigma, H3393), dextran sulfate (150 pg/mL, Sigma D8906) or galactose (100 mM, Sigma G0750) were added in the binding buffer. To detect ITLN-1 binding to coated mucins, an anti-FLAG HRP-conjugated antibody (ThermoFisher, MA1-91878-HRP) was incubated for 1 hour. The signal was detected using TMB substrate (SeraCare, 5120-0047) and the absorbance was read with a plate reader at 450 nm. For binding assays with heparin, biotin-conjugated heparin was used (Millipore, 375054) at 10 pg/mL. To detect heparin binding to coated mucins, plates were incubated with ExtrAvidin-alkaline phosphatase (Sigma, E2636) for 1 hour. The signal was revealed using the phosphatase substrate (Sigma, S0942) and the absorbance was read with a plate reader at 405 nm. The control conditions for ITLN-1 and heparin assays correspond to binding buffer only or to biotinylated albumin 10 pg/mL, respectively.

scRNA-seq Analysis of Bronchial Brushings

Bronchial cells were dissociated from the bronchial airway brushes from 2 asthmatic children using Bacillus lichenformis cold active protease (10 mg/ml), EDTA (0.5 mM), and EGTA (0.5 mM) at 4° C. with vortex mixing, followed by enzyme neutralization with FBS. Red blood cell lysis was performed and cells were washed twice in 0.04% BSA/PBS. Targeted capture of 8000 cells was performed using the 10× Genomics Chromium Next GEM Single Cell 3′ reagent kit chemistry. Sample capture, cDNA synthesis, and library preparation was performed following 10× Genomics Chromium Next GEM Single Cell 3′ chemistry (CG000204 Rev D), and barcoded samples were pooled and sequenced on an Illumina NovaSeq 6000.

Sequencing reads were processed with Cell Ranger (version 4) to produce gene UMI count matrix. Cells were required to have at least 500 genes expressed and 1,000 UMIs. Additionally, we removed cells with greater than 30% MT reads, resulting in 11,515 remaining cells to use for clustering analysis. Integration of the two bronchial brushing datasets was carried out with the SCtransform integration approach implemented in Seurat using the top 30 canonical correlates derived from 5,000 most variable genes across the two datasets. PCA on the integrated dataset was then carried out and used the top 30 PC dimensions for UMAP visualization and to perform SNN clustering with the Leiden algorithm (resolution=0.3, k.param=20) (Traag, V. A., Waltman, L. & van Eck, N.J. From Louvain to Leiden: guaranteeing well-connected communities. Sci Rep 9, 5233 (2019)), resulting in 17 cell clusters. These cell clusters were then assigned a cell type label based on the list of the marker genes produced with Seurat FindMarkers function.

### RNA-Seq Analysis of IL13-Stimulated Bronchial ALI Cultures

Ion AmpliSeq Transcriptome Human Gene Expression RNA-sequencing (RNA-seq) libraries (ThermoFisher) were constructed with 100 ng of RNA per sample. Barcoded libraries were pooled and sequenced on the Ion Torrent Proton sequencer using P1 chips. Sequencing reads were mapped to AmpliSeq transcriptome target regions with the torrent mapping alignment program and quantified with the Ion Torrent AmpliSeq RNA plugin using the unique mapping option. Duplicated sequences were removed from the FASTA file, and incorrect amplicon locations were corrected (Poole, A., et al. Dissecting childhood asthma with nasal transcriptomics distinguishes subphenotypes of disease. J Allergy Clin Immunol 133, 670-678 e612 (2014)). All samples were down-sampled to 8×10⁶ reads for consistency of analysis across the study.

### Whole Transcriptome Analysis of the GALA II Study Nasal Brushes

Primary nasal airway epithelial brushes were obtained from Puerto Rican children recruited as a part of the Genes-environments and Admixture in Latino Americans II (GALA 11) childhood asthma study and placed into RLT Plus lysis buffer supplemented with 40 mM DTT for RNA and DNA extraction using the AllPrep DNA/RNA MiniPrep Kit (Qiagen). RNA Normalization, library construction using KAPA Stranded mRNA-Seq Kit with KAPA mRNA Capture Beads (KAPA Biosystems), and library pooling for a total of 695 subjects were all performed on the Beckman Coulter FXP automation system. Briefly, RNA samples were randomized over the normalization plate in alternating control and asthmatic conditions at 250 ng per well. Illumina Dual-Index adapters (Integrated DNA Technologies) were used to barcode libraries using 12 cycles of amplification. Samples were pooled using equal concentrations of six libraries/pool and pair-end sequenced with Illumina HISEQ® 2500 System sequencing. Sequencing reads were trimmed with Skewer (—end-quality 15, —mean-quality 25, —min 30) (Jiang, H., Lei, R., Ding, S. W. & Zhu, S. Skewer: a fast and accurate adapter trimmer for next-generation sequencing paired-end reads. BMC Bioinformatics 15, 182 (2014)). Trimmed reads were aligned and mapped to human reference genome GRCh38 with GSNAP (Wu, T. D. & Nacu, S. Fast and SNP-tolerant detection of complex variants and splicing in short reads. Bioinformatics 26, 873-881 (2010)). Transcript quantification of these aligned reads on iGenomes GTF file were conducted with HTSeq to generate a gene count matrix (Anders, S., Pyl, P. T. & Huber, W. HTSeq—a Python framework to work with high-throughput sequencing data. Bioinformatics 31, 166-169 (2015)). Additionally, Kallisto was used to generate Transcript per Million (TPM) values for all the subjects (Bray, N. L., Pimentel, H., Melsted, P. & Pachter, L. Near-optimal probabilistic RNA-seq quantification. Nat Biotechnol 34, 525-527 (2016)). A total of 17,039 genes with TPM>0.1 and read counts>6 for at least 20% of the subjects were subjected to downstream eQTL analysis. Differential expression analysis of ITLN1 expression between genotype groups stratified by type 2 status was performed with DESeq2 (Love, M. I., Huber, W. & Anders, S. Moderated estimation of fold change and dispersion for RNA-seq data with DESeq2. Genome Biol 15, 550 (2014)). GALA II RNA-seq data used in this study has been previously deposited in the National Center for Biotechnology Information/Gene Expression Omnibus (GEO) GSE152004.

### Genotype Analysis for GALA II Study

Blood samples were collected from subjects in the Genes-environments and Admixture in Latino Americans study (GALA II) asthma study, and a total of 681 were included in this analysis. The genotype and phenotype information analyzed in this manuscript is publicly available via dbGAP (dbGAP accession numbers: phs001274.v1.p1, phs001180.v1.p1, and phs00921.v1.p1). Samples were genotyped with Affymetrix Axiom LAT 1 (World Array 4) and LAT plus HLA genome-wide arrays (Affymetrix, Santa Clara, CA). SNPs were removed if they failed manufacturer's quality control, had genotyping call rates below 95%, and/or had a deviation from Hardy-Weinberg equilibrium (p<10⁻⁶) within controls. Samples were filtered if there is a discrepancy between genetic sex and reported gender. When cryptic relatedness (PI_HAT>0.3) was detected for a pair of samples, one of them was removed.

GALA II ITLN1 eQTL Analysis

Expression quantitative trait loci (eQTL) analysis was performed following a pipeline similar to the Genotype-Tissue Expression (GTEx) V7 protocol (Consortium GT, L. D., Coordinating Center—Analysis Working G, Statistical Methods groups-Analysis Working G, Enhancing Gg, Fund NIHC, et al. Genetic effects on gene expression across human tissues. Nature. 2017; 550(7675):204-13) using paired genotype-gene expression data from 681 GALA II donors. After initial filtering on minor allele frequency (>0.01), minor allele subject count (>10), a total of 8,432 SNPs within 1 Mb of ITLN1 TSS were tested for association with ITLN1 gene expression level. ITLN1 gene expression was TMM normalized with edgeR followed by inverse normalization (Robinson, M. D., McCarthy, D. J. & Smyth, G. K. edgeR: a Bioconductor package for differential expression analysis of digital gene expression data. Bioinformatics 26, 139-140 (2010); McCarthy, D. J., Chen, Y. & Smyth, G. K. Differential expression analysis of multifactor RNA-Seq experiments with respect to biological variation. Nucleic Acids Res 40, 4288-4297 (2012)). Probabilistic Estimation of Expression Residuals (PEER) factors were computed with R package PEER (Stegle, O., Parts, L., Piipari, M., Winn, J. & Durbin, R. Using probabilistic estimation of expression residuals (PEER) to obtain increased power and interpretability of gene expression analyses. Nat Protoc 7, 500-507 (2012)). Admixture factors were estimated with the software Admixture (Alexander, D. H., Novembre, J. & Lange, K. Fast model-based estimation of ancestry in unrelated individuals. Genome Res 19, 1655-1664 (2009)). ITLN1 eQTL mapping was performed with FastQTL (Ongen, H., Buil, A., Brown, A. A., Dermitzakis, E. T. & Delaneau, O. Fast and efficient QTL mapper for thousands of molecular phenotypes. Bioinformatics 32, 1479-1485 (2016)), and adjusted for age, gender, body mass index (BMI), asthma status, 60 PEER factors, and four admixture factors. A stepwise regression approach implemented in QTLtools (Delaneau, O., et al. A complete tool set for molecular QTL discovery and analysis. Nat Commun 8, 15452 (2017)) was used to identify independent ITLN1 eQTLs.

### Sputum Induction and Processing

Subjects inhaled nebulized 3% saline through a mouthpiece for 12 minutes, and interrupted inhalation at 2-minute intervals to spit saliva into a saliva cup and induced sputum into a sputum cup (Gershman, N. H., Wong, H. H., Liu, J. T., Mahlmeister, M. J. & Fahy, J.V. Comparison of two methods of collecting induced sputum in asthmatic subjects. Eur Respir J 9, 2448-2453 (1996)). Saliva was discarded, and induced sputum was processed. A 10% solution of Sputolysin (EMD Millipore) was added at a 1:1 g/ml (sputum weight/Sputolysin) ratio to the induced sputum, mixed with a serologic pipette, and placed in a 37° C. shaking water bath for 15 minutes. Samples were mixed at intervals, total and differential cell counts were determined. The sample was then centrifuged at 4° C. at 2,000 rpm for 10 minutes. The cell pellet was resuspended in 1 ml of Qiagen RNAprotect Saliva Reagent (Qiagen). Cell pellets were stored at −80° C., and all RNA was shipped to the UCSF Sputum Core for RNA extraction using the RNeasy Qiagen kit (Qiagen), as previously described (Peters, M. C., et al. Refractory airway type 2 inflammation in a large subgroup of asthmatic patients treated with inhaled corticosteroids. J Allergy Clin Immunol 143, 104-113 e114 (2019); Peters, M. C., et al. Measures of gene expression in sputum cells can identify TH2-high and TH2-low subtypes of asthma. J Allergy Clin Immunol 133, 388-394 (2014)). RNA concentration and quality were measured with the Agilent 2100 bioanalyzer, and samples with an RNA integrity number <5 were considered degraded and excluded from analysis.

### SARP Study Genotyping

DNA from subjects enrolled in the Severe Asthma Research Program (SARP3) study was genotyped using the Illumina Multi-Ethnic Global BeadChip (MEGA) Chip as previously described (Cardet, J. C., et al. Clinical and molecular implications of RGS2 promoter genetic variation in severe asthma. J Allergy Clin Immunol (2022)).

### Sputum Whole-Transcriptome RNA Sequencing

KAPA mRNA HyperPrep (Roche) whole-transcriptome libraries were constructed with 20 ng RNA input per sample, barcoded with Illumina Dual Index Adapters and amplified for 16 cycles. Completed libraries were pooled together by concentration and sequenced using the Illumina NovaSeq 6000 system. Raw sequencing reads were trimmed using skewer (Jiang, H., Lei, R., Ding, S. W. & Zhu, S. Skewer: a fast and accurate adapter trimmer for next-generation sequencing paired-end reads. BMC Bioinformatics 15, 182 (2014)) with parameters (end-quality=15, mean-quality=25, min=30). Trimmed reads were then aligned to the human reference genome GRCh38 using HiSat2 with default parameters (Kim, D., Langmead, B. & Salzberg, S. L. HISAT: a fast spliced aligner with low memory requirements. Nat Methods 12, 357-360 (2015)). Gene quantification was performed with htseq-count using GRCh38 ensemble v84 gene transcript model (Anders, S., Pyl, P. T. & Huber, W. HTSeq—a Python framework to work with high-throughput sequencing data. Bioinformatics 31, 166-169 (2015)). Variance stabilization transformation implemented in DESeq2 was then performed on the raw gene count matrix to create a variance-stabilized gene expression matrix suitable for downstream analyses. Differential expression analysis of ITLN1 expression between genotype groups were performed with DESeq2 (Love, M. I., Huber, W. & Anders, S. Moderated estimation of fold change and dispersion for RNA-seq data with DESeq2. Genome Biol 15, 550 (2014)).

### Analysis of Mucus Plugging Scores

Airway lumen mucus plugs were identified in subjects enrolled in the Severe Asthma Research Program (SARP) using multidetector computed tomography (MDCT) scans, across sequential transverse CT slices, and were formally quantified as previously described (Dunican, E. M., et al. Mucus plugs in patients with asthma linked to eosinophilia and airflow obstruction. J Clin Invest 128, 997-1009 (2018)). CT scans were independently scored by radiologists using a developed visual scoring system, and the mucus plug scores were then averaged. To test for association between rs4656959 genotype and mucus plug scores, we used the negative binomial mixed model implemented in SAS (Cary, NC) software v9.04 PROC GLIMMIX (METHOD=RSPL; DDFM=KENWARDROGER2), with subjectID as random effect and adjusting for age, gender, and two admixture factors. Prior to model fitting, mucus plug scores were converted into integers using ceiling function in R.

### Weighted Gene Coexpression Network Analysis

To capture the heterogeneity of transcriptomic profiles from cultured airway epithelial, nasal airway epithelial, and airway sputum, we performed weighted gene coexpression network analysis (WGCNA) on the variance-stabilized expression matrix from each respective sample type. The gene networks from the IL-13-stimulated HBEC ALI cultures were constructed on 15,152 genes using the following parameters: softPower=10, deepSplit=2, minClusterSize=40, and PAM=F, resulting in 18 gene networks. Construction of gene coexpression networks from the GALA II nasal samples has been described elsewhere (Sajuthi, S. P., et al. Type 2 and interferon inflammation regulate SARS-CoV-2 entry factor expression in the airway epithelium. Nat Commun 11, 5139 (2020)). Briefly, WGCNA was run on 17,473 expressed genes with softPower=9, minClusterSize=20, deepSplit=2, and PAM=T. Similarly, WGCNA was performed on 18,197 expressed genes from the SARP sputum samples with softPower=12, minClusterSize=10, deepSplit=1, and PAM=T. To classify type 2 status, first we hierarchically clustered longitudinal SARP samples using ward. D2 and the Pearson correlation distance metric using the normalized expression of type 2 module genes and used the first split of the constructed dendrogram to classify samples into type 2-low and type 2-high groups. A subject was classified as type 2-high if any of its associated longitudinal samples belonged to the type 2-high group, otherwise they were classified as type 2-low.

### Example 2: IL-13 Mucus Metaplastic Epithelia Produce a Mucus Layer Containing ITLN-1

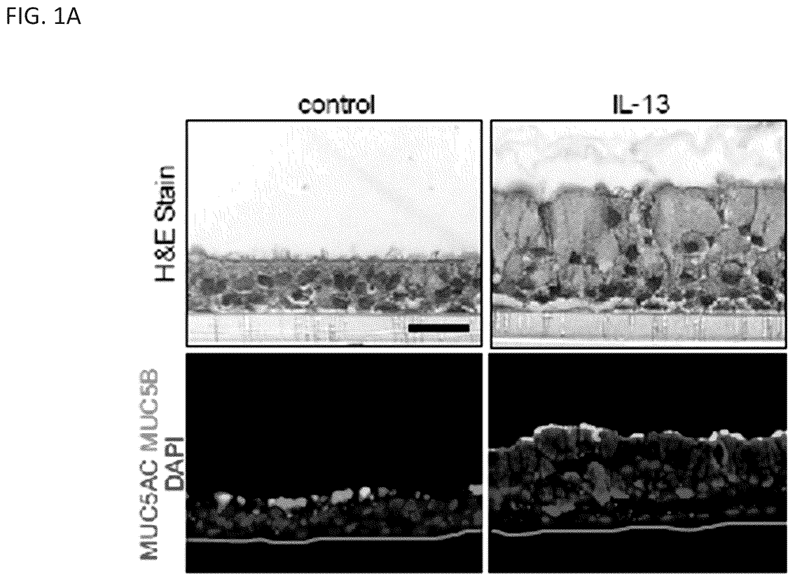

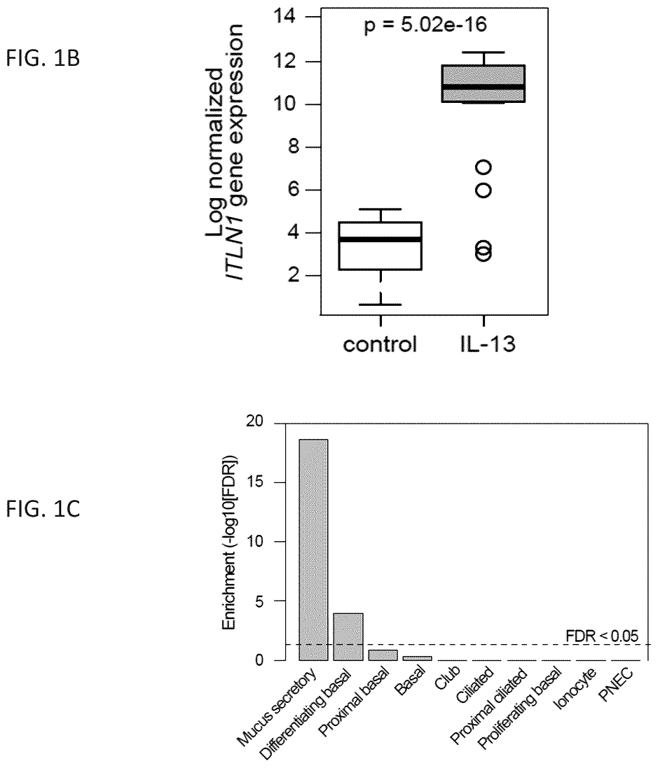

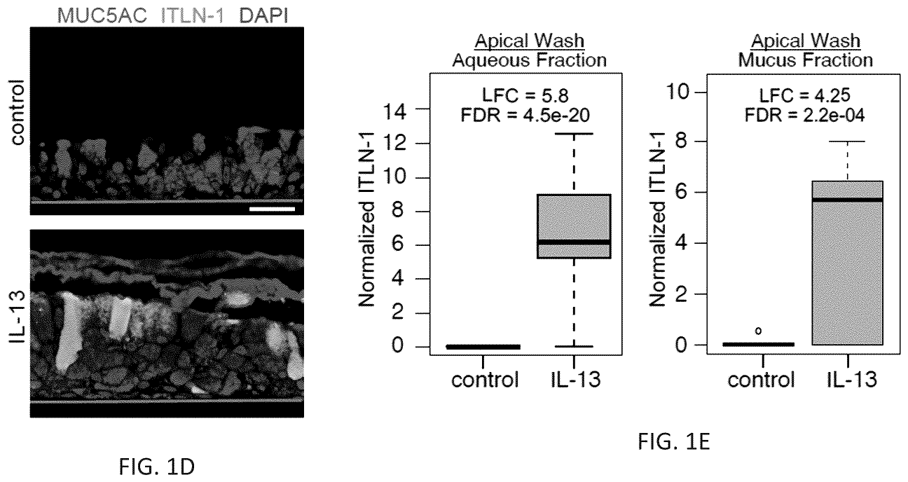

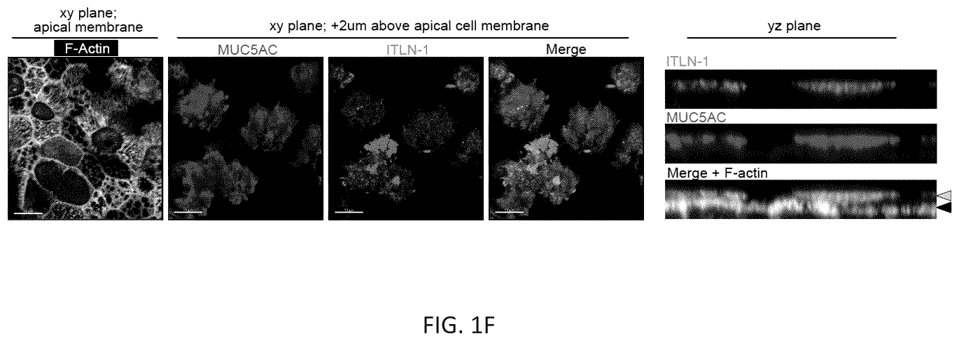

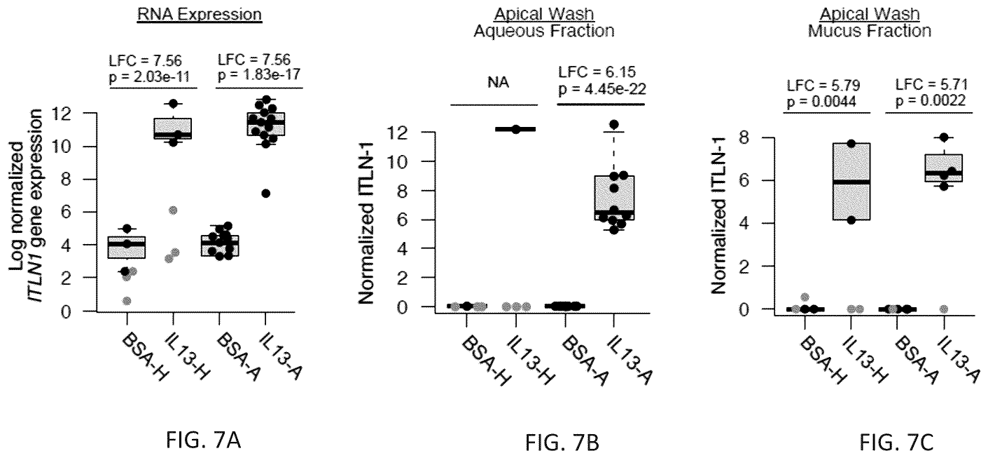

To investigate airway epithelial regulation and function of ITLN-1 mucociliary airway epithelial air-liquid interface (ALI) cultures from primary human bronchial epithelial basal stem cells (HBEC) of 13 asthmatic and 6 healthy control donors was generated. To model the effects of airway type 2 inflammation on ITLN-1, paired cultures were chronically stimulated (culture days 11-21) with interleukin 13 (IL-13), which resulted in mucus metaplasia (FIG. 1A). RNA-seq expression profiling revealed ITLN1 was poorly expressed in control cultures, but was upregulated 170-fold among IL-13 mucus metaplastic cultures (FDR=5.0e-16; FIG. 1). No differences in either baseline or IL-13 induced ITLN1 expression were observed between cultures derived from healthy versus asthmatic donors (FIG. 7A). To better understand the cellular and mechanistic context of ITLN1 induction by IL-13, co-expression network analysis on all expressed, variable genes was performed, identifying 18 networks broadly representing different epithelial cellular functions and molecular pathways. ITLN1 was part of a 598 gene network, which included canonical T2 inflammation markers (POSTN, CST1, CDH26), markers of mucus secretory cells (FCGBP, CLCA1, DPP4), and established transcription factor drivers of IL-13-induced mucus metaplasia (SPDEF and FOXA3). Formal enrichment analysis of the ITLN1 network genes using GO, KEGG, and Reactome databases identified pathways involved in mucin formation and mucus secretion, including ion transport and o-linked glycosylation of mucins. Moreover, these ITLN1 network genes were strongly enriched (FDR 2.3e-19) for marker genes of mucus secretory cells based on human lung scRNA-seq data (FIG. 1C) (Travaglini, K. J., et al. A molecular cell atlas of the human lung from single-cell RNA sequencing. Nature 587, 619-625 (2020)).

Next whether ITLN-1 protein was localized to mucus secretory cells, as suggested by the gene expression network analysis was evaluated. Strikingly, control cultures, which predominantly express MUC5B protein was found to have no observable ITLN-1 protein, despite expressing a low, but measurable level of ITLN1 mRNA. In contrast, prominent cytoplasmic ITLN-1 protein was found to be present in IL-13 cultures, specifically among a subset of MUC5AC⁺ mucus secretory cells (FIG. 1D). Mass spectrometry-based proteomic analysis of culture-derived apical secretions detected ITLN-1 peptides in both the aqueous and mucus secretions of the IL-13 treated cultures, while ITLN-1 peptides were not detected in control culture secretions (FIG. 1E). ITLN-1 protein secretion did not differ significantly between cultures derived from healthy versus asthmatic donors (FIGS. 7B and 7C). The presence of ITLN-1 in airway mucus was confirmed using confocal imaging of immunofluorescent (IF) labeled IL-13 cultures, where ITLN-1 protein was found interspersed throughout the epithelial mucus layer (FIG. 1F). Together, these results demonstrate that ITLN-1 gene and protein expression are specifically induced by IL-13-driven mucus metaplastic airway epithelia, with ITLN-1 protein being apically secreted and integrated into mucus.

### Example 3: Loss of ITLN-1 Protein Partially Restores IL-13-Mediated Mucostasis

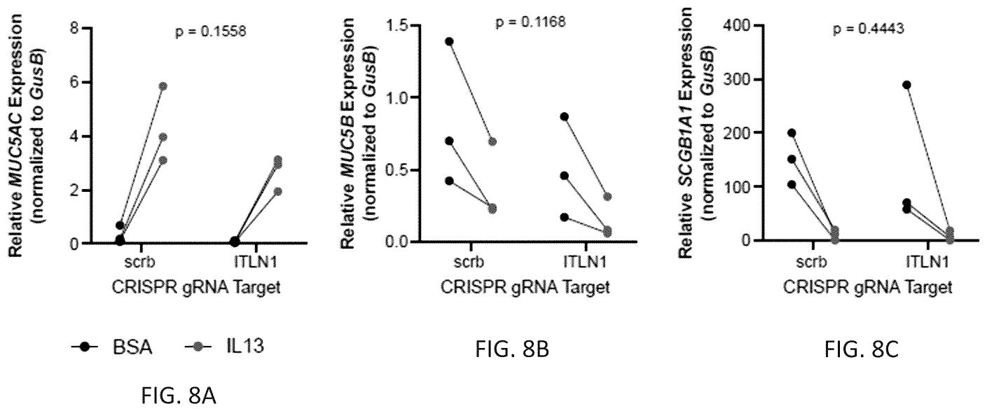

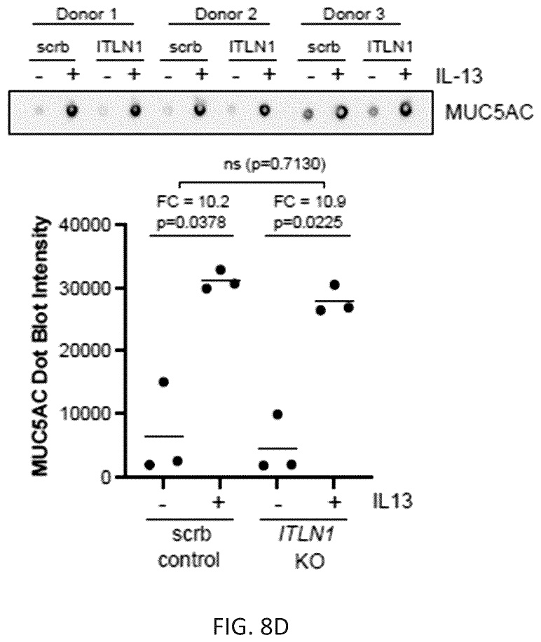

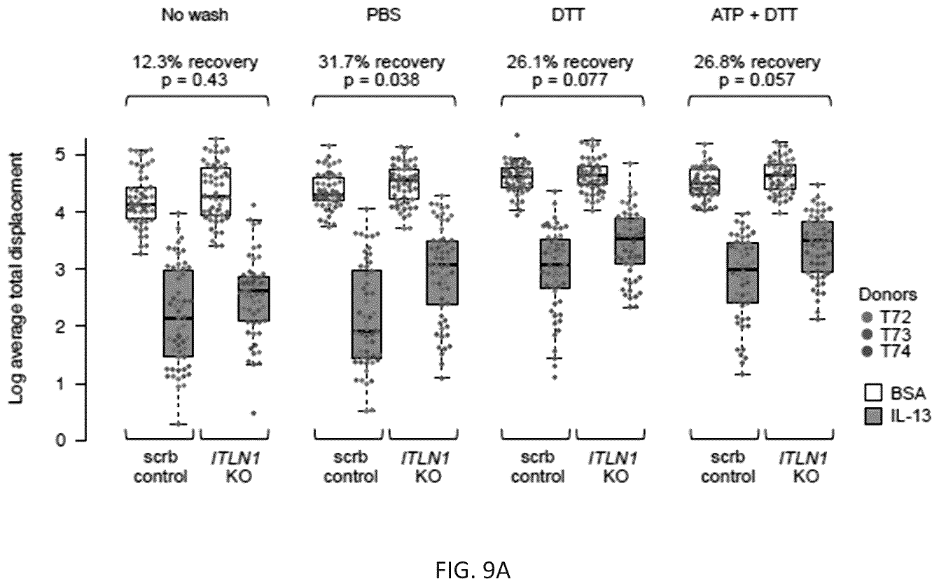

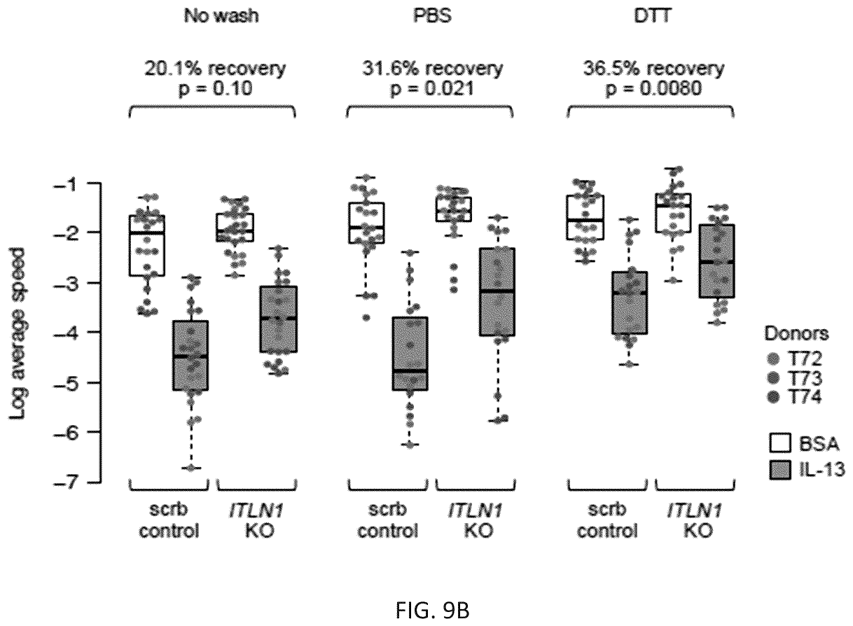

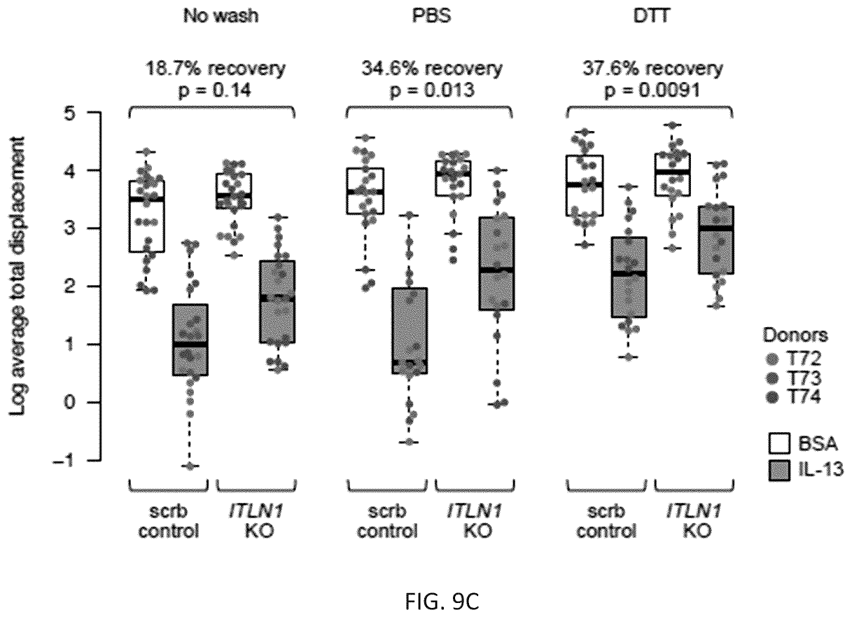

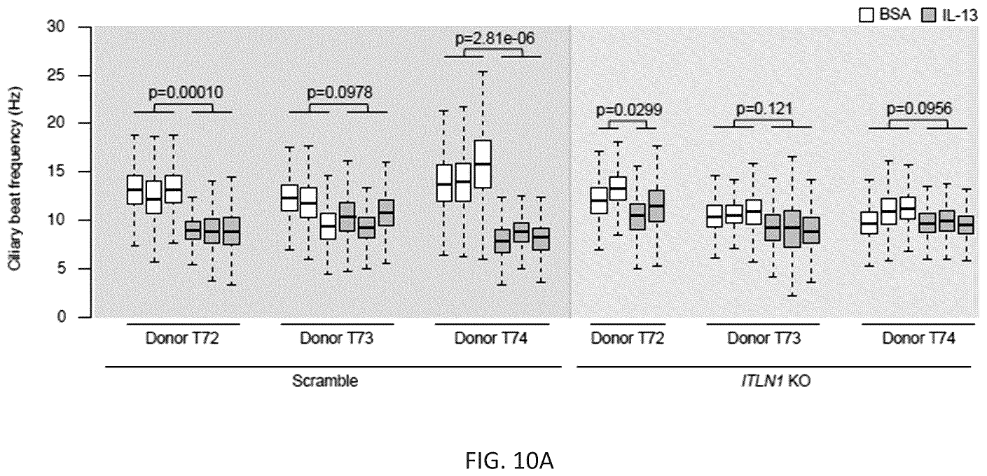

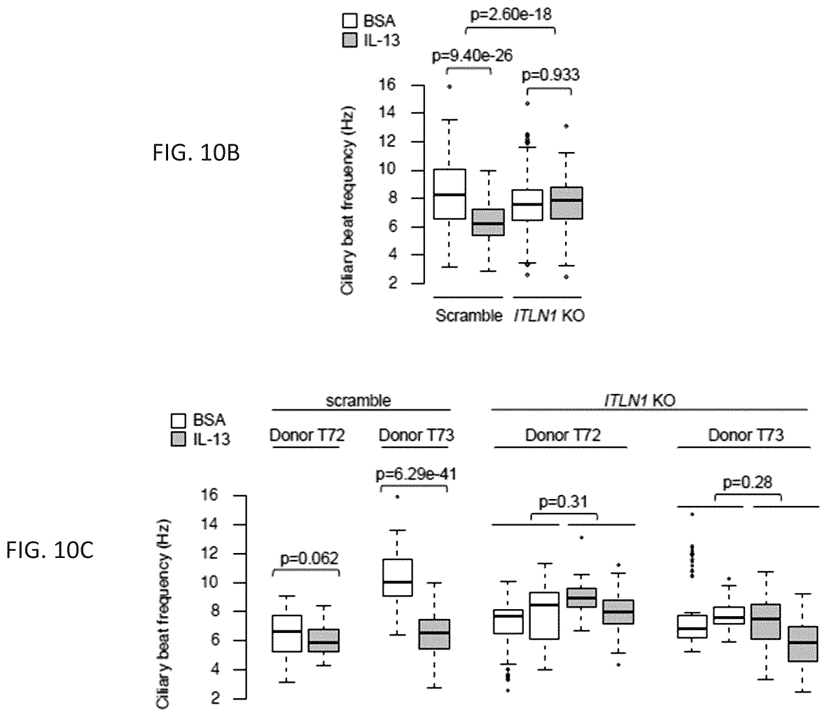

Given the differential presence of ITLN-1 in the mucus of control versus IL-13 metaplastic epithelial cultures, the inventors hypothesized that IL-13-induced mucostasis could be mediated by ITLN-1. Generation of ITLN-1 knockout tracheal airway epithelial basal cells through CRISPR-Cas9 targeting of the ITLN1 gene resulted in highly efficient editing of genomic DNA at the ITLN1 target site as measured by high resolution melt curve analysis (FIG. 2A). Additionally, high indel rates of 76%, 79%, and 82% at the ITLN1 target site across the 3 edited donors were achieved, as measured by Inference of CRISPR Edits (ICE) analysis. These ITLN1-targeted and mock-targeted basal cells were used to generate mucociliary ALI cultures for functional evaluation. ITLN1-targeted cultures treated with IL-13 exhibited a 96.4% loss of apically secreted ITLN-1 protein as compared to the IL-13-treated mock-targeted cultures (FIG. 2B). ITLN1 KO was not associated with changes in the expression of secretory genes known to be modified by IL-13, including MUC5AC, MUC5B, or SCGBIA1 (FIG. 8A-8C). Moreover, IL-13-induced secretion of MUC5AC was not altered by ITLN1 KO (FIG. 8D), indicating that similar mucociliary differentiation and IL-13 responses were achieved by the ITLN1 KO versus mock-edited mucociliary cultures. Mucociliary movement (MCM) among mock-targeted and ITLN1 KO cultures were then assessed, with and without IL-13 treatment, using video-based fluorescent bead tracking. These MCM measurements were performed on cultures prior to washing the mucus layer, after a PBS wash (to disrupt the aqueous mucus components), following a PBS-DTT wash (to disrupt mucus disulfide protein bonds), and after ATP stimulation (to induce release of intracellular mucin granules) followed by PBS-DTT wash. As expected, among mock-edited cultures, IL-13 treatment decreased particle speed in unwashed (2.80-fold), PBS washed (3.80-fold), PBS-DTT washed (3.61-fold), and ATP+PBS-DTT washed cultures (3.36-fold) (FIG. 2C), with similar results observed for particle displacement (FIG. 9A). However, among ITLN1 KO cultures it was found that 11% (unwashed, p=0.48), 28% (PBS washed, p=0.036), 22% (PBS-DTT washed, p=0.11), and 27% (ATP+PBS-DTT washed, p=0.037) of this IL-13-induced reduction in particle speed was restored (FIG. 2C). Similar results were also found for particle displacement (FIG. 9A). Direct evaluation of cilia movement through the measurement of ciliary beat frequency (CBF) in these cultures demonstrated that IL-13 treatment resulted in a 31% reduction in CBF in control cultures (p-value=1.62e-08; FIG. 2D and FIG. 10A). In contrast, it was found that only a 12% reduction in CBF induced by IL-13 (p=0.023) among the ITLN1 KO mucociliary cultures, constituting a 67.5% reduction in the effect of IL-13 in ITLN-1 KO cultures (p=0.0012). An independent regeneration of mucociliary epithelia from these donor ITLN1 KO and mock-edited basal cells, followed by MCM (no ATP stimulation performed) and CBF assays achieved similar results (FIGS. 9B-9C; FIGS. 10B-10C). Altogether, these data suggest that apically secreted ITLN-1 protein plays a consequential role in the mucostatic effects that occur in the airway epithelium under IL-13-induced inflammation.

### Example 4: ITLN-1 Binds MUC5AC Through Electrostatic Interactions

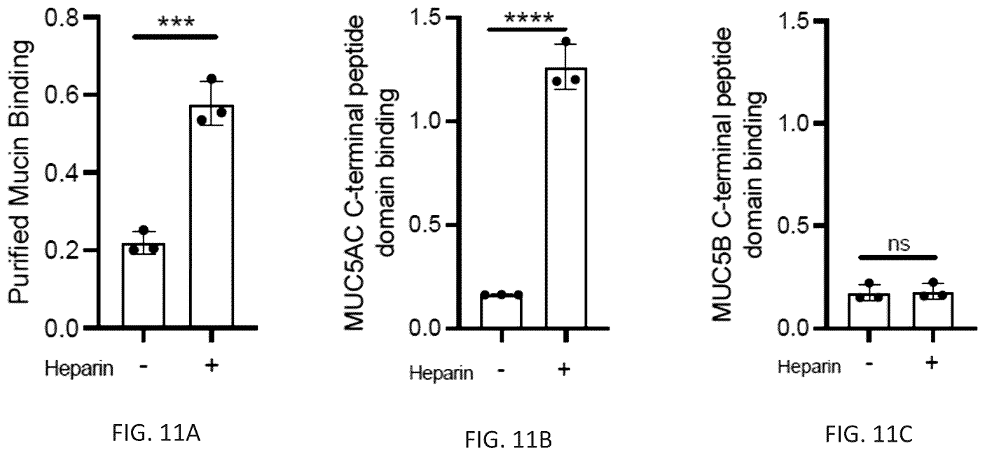

Based on the MCM changes observed in the ITLN-1 KO, the inventors hypothesized that ITLN-1 protein might bind to mucin monomers resulting in mucin cross-linking and thus alter the visco-elastic properties of mucus. To determine this, the binding of recombinant human ITLN-1 protein to immobilized mucin proteins purified from the airway mucus of five donors was evaluated. This assay indicated significant binding between ITLN-1 and the purified mucins (FIG. 3A). Considering that ITLN-1 has high affinity for galactofuranosyl residues, the ability of galactose to inhibit this binding was evaluated and found no significant inhibition of the ITLN-1-mucin interaction with the addition of galactose (FIG. 3A), confirming previous works showing that ITLN-1 has no affinity for mammalian glycans (Wesener, D. A., et al. Recognition of microbial glycans by human intelectin-1. Nat Struct Mol Biol 22, 603-610 (2015); Tsuji, S., et al. Human intelectin is a novel soluble lectin that recognizes galactofuranose in carbohydrate chains of bacterial cell wall. J Biol Chem 276, 23456-23463 (2001). As ITLN-1 has a prominent negative charge, the inventors considered whether charge-charge interactions could mediate this binding. Testing ITLN-1 binding to mucin protein in the presence of heparin or dextran sulfate, two compounds with high negative charge density, revealed that both reagents effectively decreased ITLN-1 binding to purified mucin proteins by 51.9% and 74.0%, respectively (FIG. 3A). Previously, heparin has been shown to bind the C-terminal tail of the intestinal mucin MUC2 (Xu, G., Forstner, G. G. & Forstner, J. F. Interaction of heparin with synthetic peptides corresponding to the C-terminal domain of intestinal mucins. Glycoconj J 13, 81-90 (1996); Xu, G., Bell, S. L., McCool, D. & Forstner, J. F. The cationic C-terminus of rat Muc2 facilitates dimer formation post translationally and is subsequently removed by furin. Eur J Biochem 267, 2998-3004 (2000)), and a similar domain is present in the C-terminal tail sequences of both the MUC5AC and MUC5B airway mucin proteins. Therefore, the inventors tested the ability of heparin to bind to human purified mucins, and detected significant interaction between heparin and the human purified airway mucins (FIG. 11A). Next, the binding of heparin and ITLN-1 to immobilized C-terminal peptides of MUC5AC and MUC5B was tested and it was found that individually both ITLN-1 and heparin bound to C-terminal MUC5AC but not C-terminal MUC5B (FIG. 3B, FIGS. 11B-11C). Furthermore, the ITLN-1 C-terminal MUC5AC interaction was significantly inhibited by the addition of heparin, suggesting this interaction is electrostatic in nature (FIG. 3B). Together, these data support significant binding between ITLN-1 and airway MUC5AC, presenting the trimeric form of ITLN-1 as a potential crosslinker of mucins in pathologic airway mucus, and as a potential mechanism driving the ITLN-1 effects on mucociliary functions (FIG. 3C).

### Example 5: ITLN1 Upregulation in T2-High Asthmatics is Strongly Dependent on an eQTL Variant

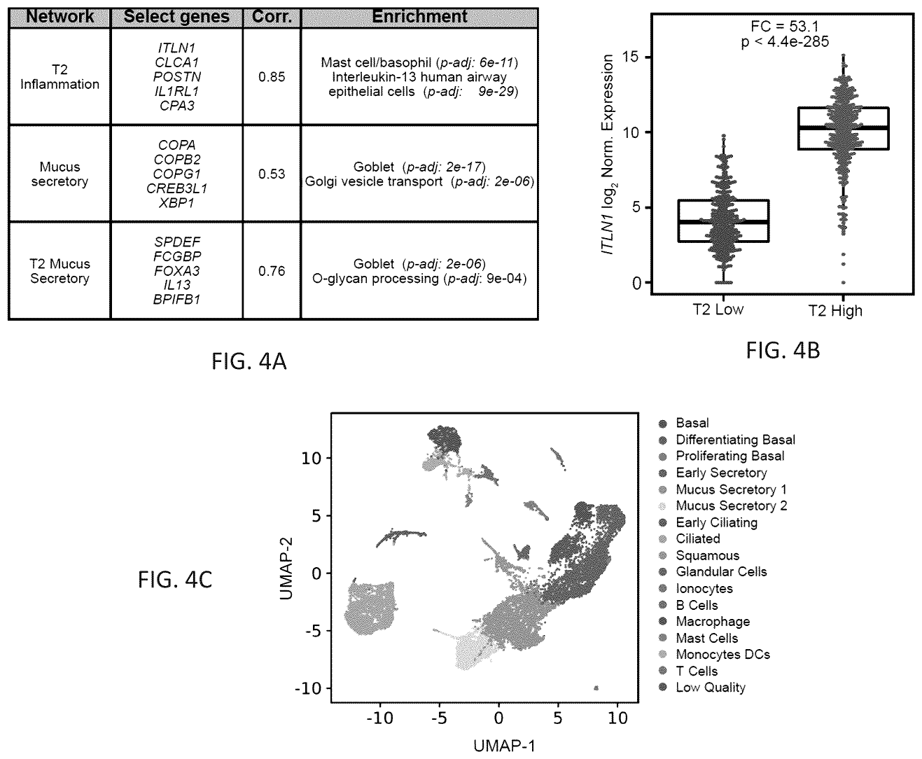

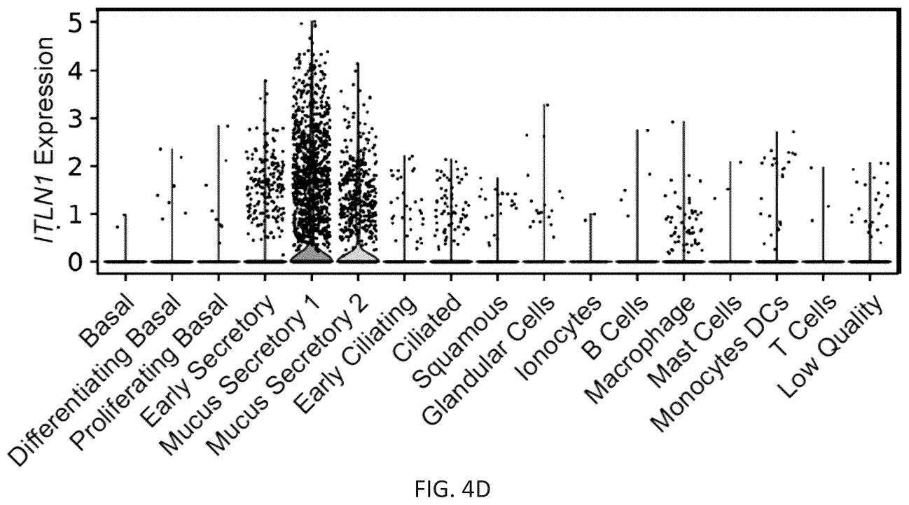

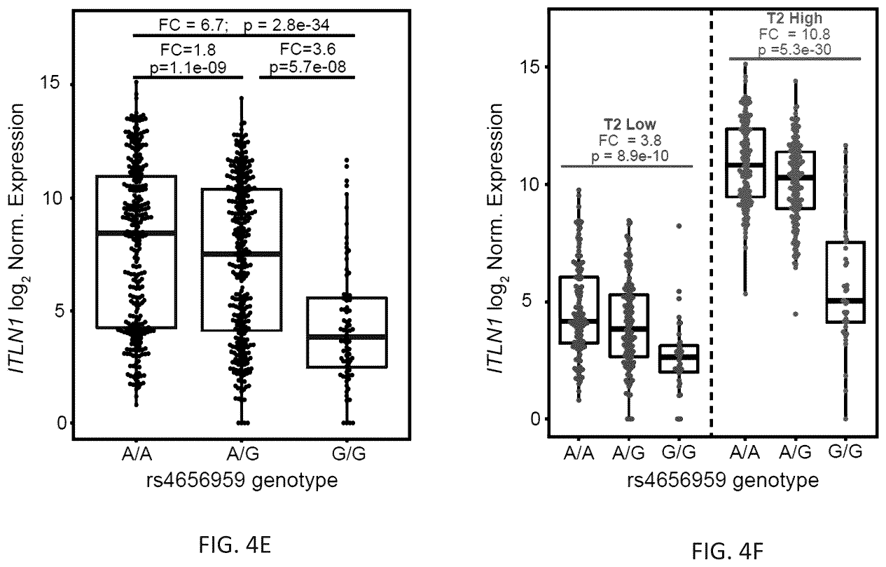

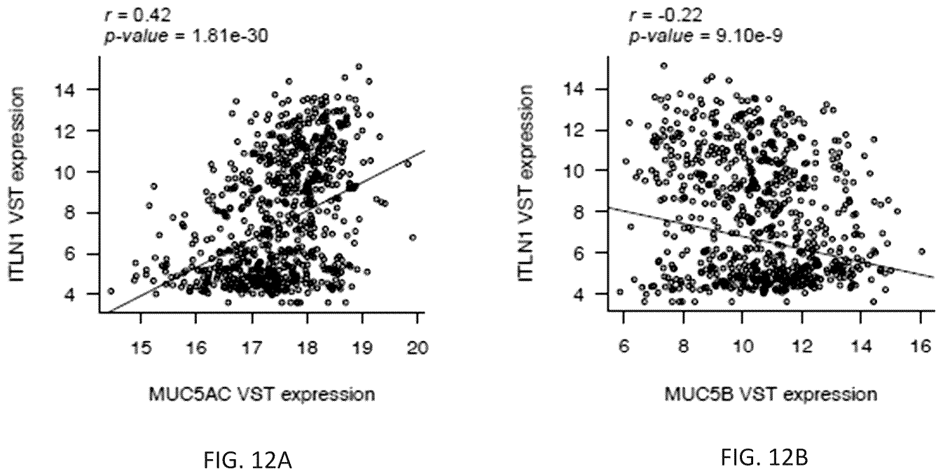

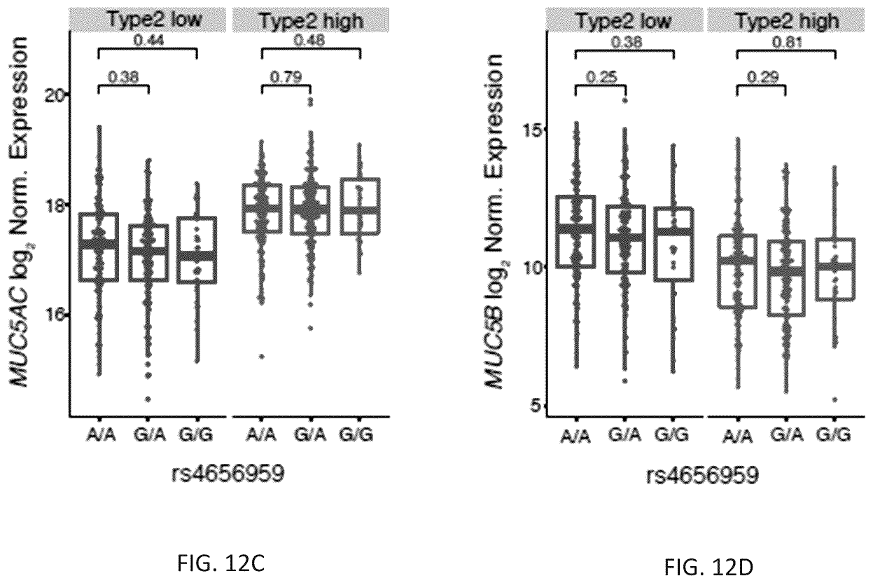

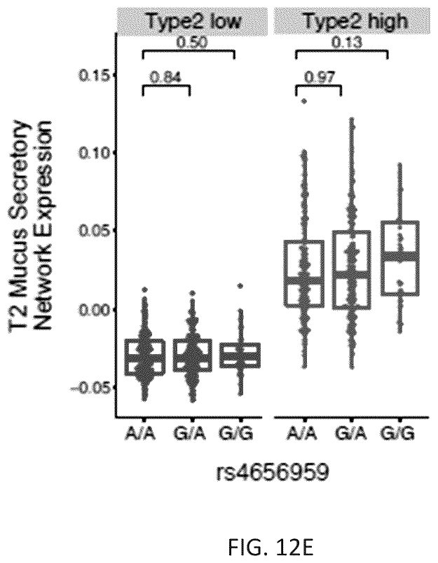

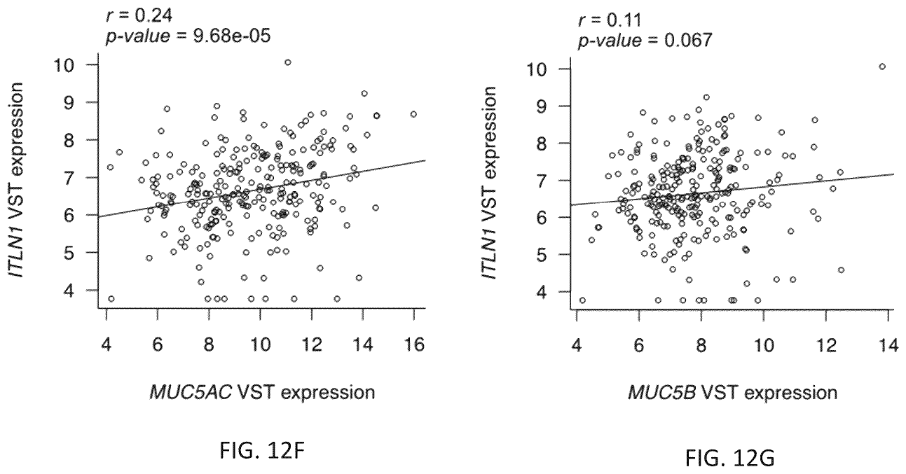

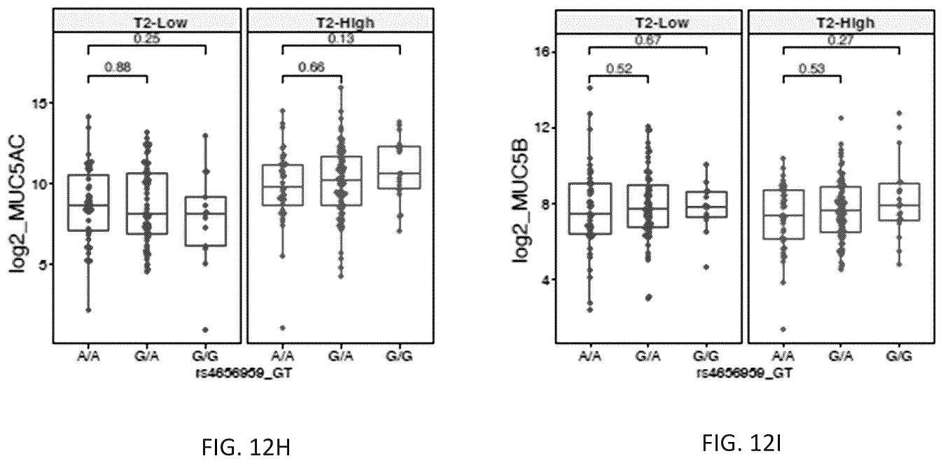

Examination of in vivo regulation of ITLN1 airway expression was undertaken. To do this published network gene expression data derived from RNA-seq data generated on the in vivo nasal airway epithelial brushings of 695 children in the Genes-Environments and Admixture in Latino Americans (GALA II) asthma study (Sajuthi, S. P., et al. Type 2 and interferon inflammation strongly regulate SARS-CoV-2 related gene expression in the airway epithelium. bioRxiv (2020); Sajuthi, S. P., et al. Nasal airway transcriptome-wide association study of asthma reveals genetically driven mucus pathobiology. Nat Commun 13, 1632 (2022)) was analyzed. These children were assigned into one of two groups, T2-high (n=364) and T2-low (n=331), by hierarchically clustering (K=2) subjects based on their expression of the identified T2 inflammatory network(Sajuthi, S. P., et al. Type 2 and interferon inflammation strongly regulate SARS-CoV-2 related gene expression in the airway epithelium. bioRxiv (2020); Sajuthi, S. P., et al. Nasal airway transcriptome-wide association study of asthma reveals genetically driven mucus pathobiology. Nat Commun 13, 1632 (2022)). This T2 inflammatory network contained the canonical T2 epithelial biomarker genes, POSTN, CLCA1, CPA3, CCL26, DPP4, and ILIRL1. Importantly, it was determined that the ITLN1 gene was in this T2 inflammation network, and its expression was highly correlated with overall T2 network expression (r=0.85, p=1.5e-191; FIG. 4A). In fact, ITLN1 expression was 53.1-fold higher among T2-high vs. T2-low subjects (p<4.4e-285; FIG. 4B). Additionally, it was determined that ITLN1 expression was highly correlated with that of two mucus secretory networks (FIG. 4A), and observed a strong positive correlation between ITLN1 and the T2-associated mucin MUC5AC (r=0.42, p=1.8e-30; FIG. 12A) and a moderate negative correlation between ITLN1 and MUC5B expression (r=−0.22; p=9.1e-9; FIG. 12B). This data is consistent with the inventors in vitro data showing ITLN1 is a mucus secretory cell gene, more associated with MUC5AC expressing cells.

To confirm the airway epithelial cell types expressing ITLN1 in vivo, a single cell RNA-seq (scRNA-seq) analysis of bronchial airway epithelial brushings from two children with asthma was performed. Performing shared nearest-neighbor clustering of the 11,515 cells, 17 distinct cell clusters were identified and then assigned cell types to these clusters by overlaying known epithelial cell type marker genes with differentially expressed genes for each cell cluster (FIG. 4C). This analysis identified two mucus secretory cell populations, both of which highly expressed ITLN1, compared to all other cell populations (FIG. 4D). These results confirm and extend to the in vivo bronchial epithelium that ITLN1 is highly expressed by mucus secretory cells.

Next the role of population genetic variation in regulating airway ITLN1 expression through examination of ITLN1 eQTL data extracted from the results of a published genome-wide nasal airway cis-eQTL analysis performed on the same children in the GALA II study²¹ was evaluated. This analysis identified 523 genetic variants that were significantly associated with ITLN1 expression, which were reduced by stepwise forward-backward regression analysis to two variants with independent effects on ITLN1 expression. The variant with the most significant effect on expression (rs4656959, p=2.0e-47, MAF 0.34)), is located 4 kb upstream of the ITLN1 gene, and marked a linkage disequilibrium block of 79 variants (with r²>0.7). This ITLN1 rs4656959 variant resulted in a dramatic 6.7-fold decrease in expression between the AA and GG genotypes (p=2.8e-34; FIG. 4E). Multivariable modeling of ITLN1 expression as a function of T2 inflammation status and rs4656959 genotype was performed to investigate the independent and interactive effects of these factors. This analysis confirmed strong effects of both the rs4656959 variant (p=9.5e-08) and T2 status (p=1.5e-24; Table 1) on ITLN1 expression. Additionally, the magnitude of the genotype effect was found to be dependent on T2 inflammation (interaction p=3.1e-04), as the ITLN1 expression between AA versus GG genotype subjects was 3.8-fold lower among T2-low subjects (p=8.9e-10) but was 10.8-fold lower among T2-high subjects (p=5.3e-30; FIG. 4F). These results reveal the strong role that both T2 inflammation and the rs4656959 variant play in the regulation of ITLN1 gene expression in vivo. Moreover, the ITLN1 eQTL variant was found not associated with either MUC5AC, MUC5B, or T2 mucus secretory network expression, indicating that this loss of ITLN1 expression did not inhibit T2 inflammation-directed mucus metaplasia (FIGS. 12C-12E).

TABLE 1

| Predictor | | Partial R2 | Effect Size | | Statistics | |
|---|---|---|---|---|---|---|
| Variable | Reference | (%) | Coefficient | SE | t | p-value |
| Age | n/a | 0.11 | 0.01 | 0.01 | 1.42 | 1.55E−01 |
| Sex | Female | 0.08 | −0.06 | 0.05 | −1.17 | 2.43E−01 |
| Asthma | Healthy | 0.5 | 0.15 | 0.05 | 3 | 2.77E−03 |
| Type 2 | Low | 51.95 | 1.63 | 0.07 | 23.81 | 1.99E−91 |
| Inflammation | | | | | | |
| rs4656959 (A > G) | A/A | 8.31 | −0.28 | 0.05 | −5.39 | 9.50E−08 |
| T2 × rs4656959 | Low-A/A | 0.73 | −0.26 | 0.07 | −3.63 | 3.09E−04 |

### Example 6: The Rs4656959 Variant is Associated with Complete Loss of ITLN-1 Protein Expression and Airway Epithelial Secretion

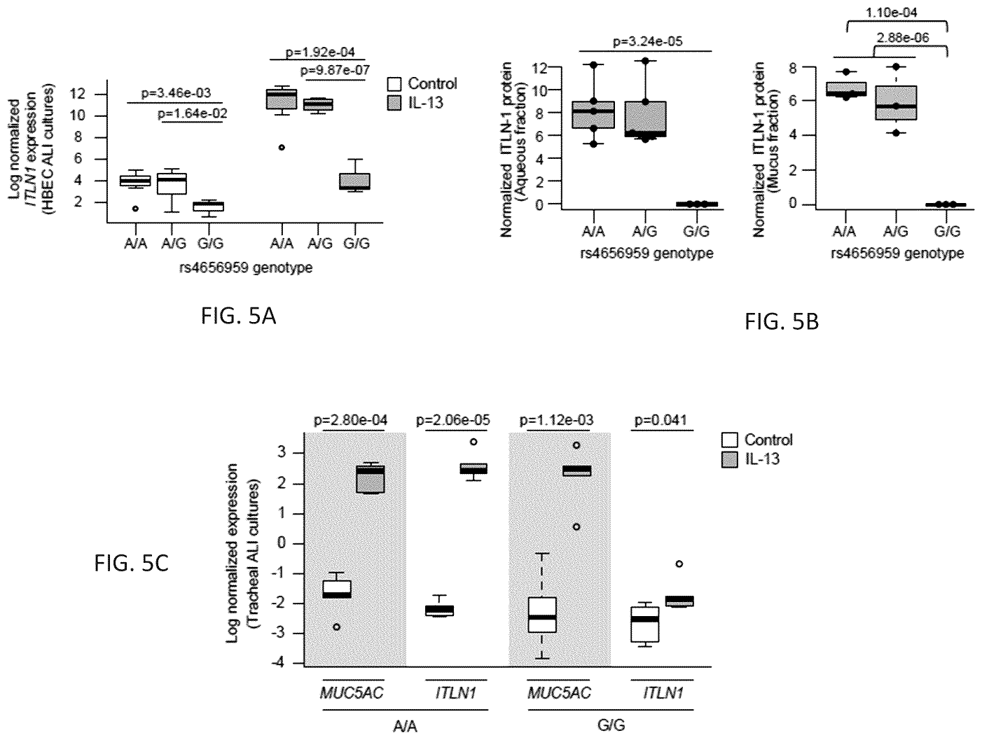

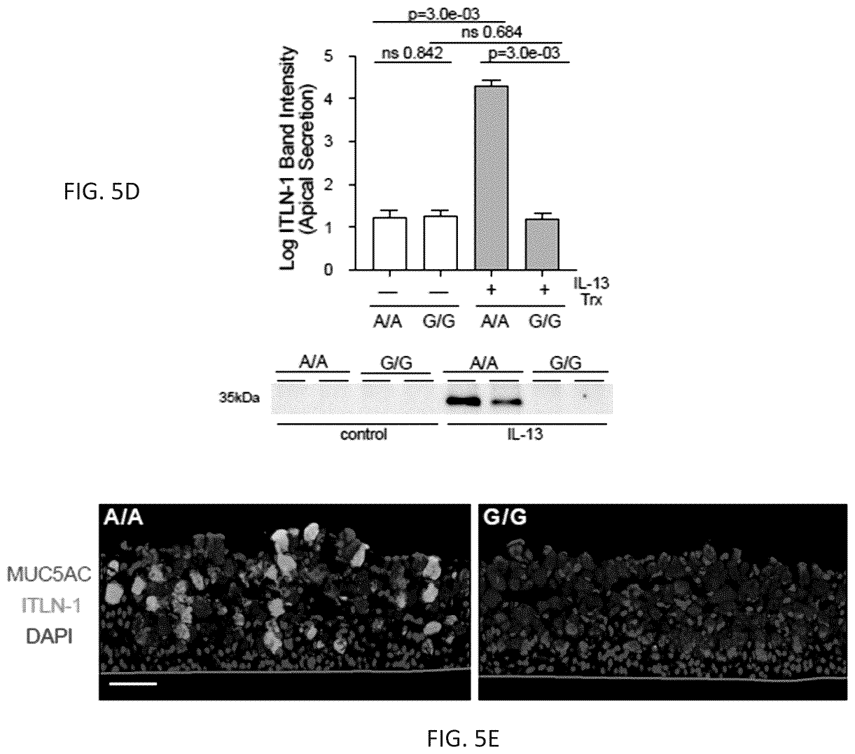

The effect of the rs4656959 variant on ITLN1 expression among the HBEC ALI cultures was genotyped and evaluated as characterized in FIGS. 1A-1F (n=19) of which 9 were the AA, 7 were the AG, and 3 were the GG genotype. A strong genotypic effect was found, with 6.9-fold lower ITLN1 expression in rs4656959 GG relative to AA genotype cultures under baseline conditions (p=3.5e-03), and 130-fold lower ITLN1 expression between genotypes when treated with IL-13 (p=1.9e-04; FIG. 5A). Furthermore, it was determined that ITLN-1 protein secretion was induced by IL-13 among both AA and AG genotype cultures, while completely absent within the aqueous or fraction collected from GG genotype cultures, with a similar result observed for the mucus fraction (FIG. 5B).

To confirm these findings, five subjects were identified each with AA and GG genotypes for the rs4656959 variant in a bank of tracheal airway epithelial basal cells. These cells were differentiated into mucociliary cultures at ALI under either control or IL-13 treatment conditions, and targeted gene expression was measured by qPCR analysis. Poor expression of ITLN1 in AA control cultures was found, which was increased 117.1-fold with IL-13, while ITLN1 expression was very minimally increased by IL-13 treatment among GG cultures (FIG. 5C). In contrast, MUC5AC, a key marker of IL-13-induced mucus metaplasia, was robustly induced among both the AA (FC=49.8, p=2.8e-04) and GG (FC=89.7, p=1.1e-03) genotype groups (FIG. 5C). Western blot analysis on apical washes from these tracheal cultures revealed that all five of the AA donors significantly upregulated apical ITLN-1 protein secretion following IL-13 stimulation (p=3.0e-03; FIG. 5D), while secreted ITLN-1 protein was absent in all control and IL-13-stimulated cultures with the GG genotype. This total absence of ITLN-1 protein among GG donors was further confirmed by immunofluorescent labeling of histological sections from these cultures (FIG. 5E). Taken together, these results demonstrate that the rs4656959 GG genotype drives the complete elimination of ITLN-1 protein, whether cellularly localized or apically secreted, even while a robust IL-13-induced mucus metaplasia is maintained.

### Example 7: The ITLN1 rs4656959 Variant is Protective Against T2-Inflammation-Driven Airway Mucus Plugging in Severe Asthmatics

Previously, the inventors determined that 58% of severe asthmatics in the Severe Asthma Research Program (SARP) cohort exhibited mucus plugging of lung segments⁸. Moreover, high mucus plug scores (>4 plugged lung segments) were associated with high sputum eosinophil levels, a clinical marker of T2 airway inflammation (Dunican, E. M., et al. Mucus plugs in patients with asthma linked to eosinophilia and airflow obstruction. J Clin Invest 128, 997-1009 (2018)). Based on these findings that ITLN-1 protein regulates mucociliary movement in an in vitro model of T2 inflammation-induced mucostasis, the inventors determined whether the rs4656959 ITLN1 eQTL can modulate risk of mucus plugging among SARP subjects.

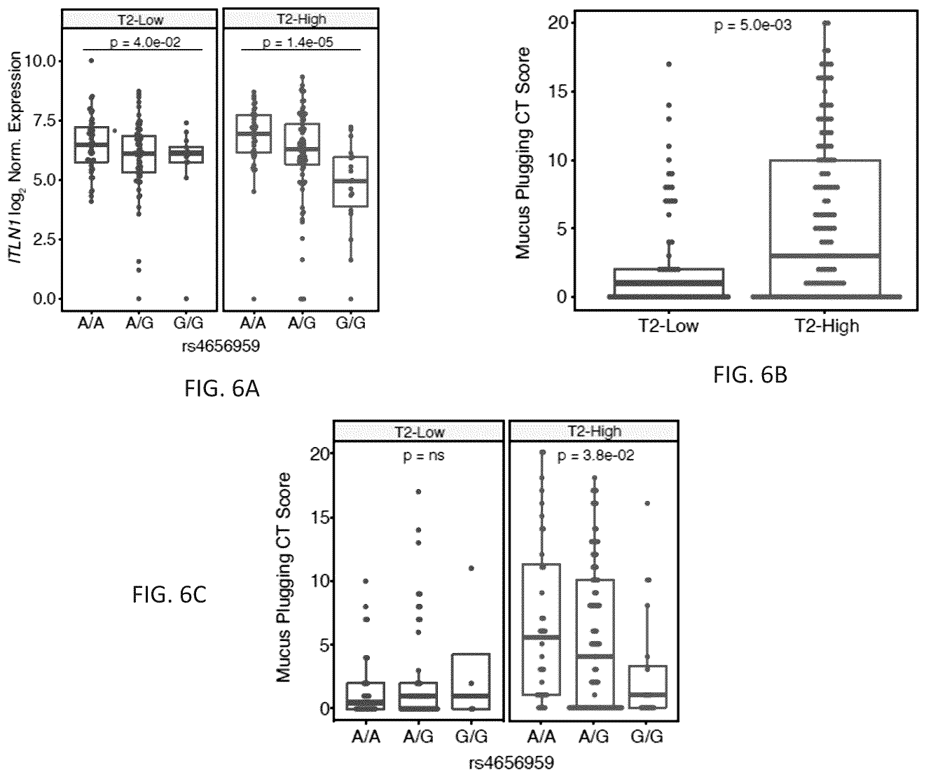

The role of both T2 inflammation and the rs4656959 variant on ITLN1 expression in lung airway samples was verified by utilizing RNA-seq data generated on sputum cell pellets from 249 severe asthmatics within the SARP cohort. Subjects were stratified into T2-high (n=132) and T2-low (n=117) groups by hierarchical clustering using expression of genes in the T2 inflammation network (see Methods). The rs4656959 variant was significantly associated with sputum ITLN1 expression among both T2-low (FC=1.8, p=4.0e-02) and T2-high subjects (FC=3.2, p=1.4e-05; FIG. 6A).), although this association was weaker, likely due the low and highly variable epithelial contribution to sputum samples, which confounds this analysis. Similar to the inventors analyses in nasal brushings in the GALA II study, no association was found between either MUC5AC or MUC5B expression and the ITLN1 eQTL (FIGS. 12H-12I). A moderate positive correlation was observed between ITLN1 and MUC5AC expression (r=0.28; p=9.4e-17; FIG. 12F), whereas ITLN1 was not correlated with MUC5B expression (r=0.04; p=0.032; FIG. 12G), reinforcing the co-expression of ITLN1 and MUC5AC.

CT-based mucus plug scores were available at two timepoints (3 years apart) on 112 SARP asthmatics. 37% of T2-low subjects exhibited a score above 0 versus 61.5% of T2-high subjects, and the mean plug score was 2.05 for T2-low subjects versus 5.22 for T2-high subjects (p=5.0e-03; FIG. 6B). With the low frequency of mucus plugs among T2-low subjects, no relationship was found between plug score and rs4656959 genotype. However, among T2-high subjects a significant additive relationship was found between the rs4656959 genotype and mucus plugging (p=3.8e-02), with the GG genotype subjects, associated with lower ITLN1 expression, exhibiting lower average mucus plug scores (AA=6.5, AG=5.3 GG=2.7; FIG. 6C).

All of the documents cited herein are incorporated herein by reference.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

### REFERENCES

1. Fahy, J.V. Type 2 inflammation in asthma—present in most, absent in many. Nat Rev Immunol 15, 57-65 (2015).
2. Wesolowska-Andersen, A. & Seibold, M. A. Airway molecular endotypes of asthma: dissecting the heterogeneity. Curr Opin Allergy Clin Immunol 15, 163-168 (2015).
3. Woodruff, P. G., et al. T-helper type 2-driven inflammation defines major subphenotypes of asthma. Am J Respir Crit Care Med 180, 388-395 (2009).
4. Sajuthi, S. P., et al. Type 2 and interferon inflammation regulate SARS-CoV-2 entry factor expression in the airway epithelium. Nat Commun 11, 5139 (2020).
5. Jackson, N. D., et al. Single-Cell and Population Transcriptomics Reveal Pan-epithelial Remodeling in Type 2-High Asthma. Cell Rep 32, 107872 (2020).
6. Peters, M. C., et al. A Transcriptomic Method to Determine Airway Immune Dysfunction in T2-High and T2-Low Asthma. Am J Respir Crit Care Med 199, 465-477 (2019).
7. Poole, A., et al. Dissecting childhood asthma with nasal transcriptomics distinguishes subphenotypes of disease. J Allergy Clin Immunol 133, 670-678 e612 (2014).
8. Dunican, E. M., et al. Mucus plugs in patients with asthma linked to eosinophilia and airflow obstruction. J Clin Invest 128, 997-1009 (2018).
9. Bonser, L. R., Zlock, L., Finkbeiner, W. & Erle, D. J. Epithelial tethering of MUC5AC-rich mucus impairs mucociliary transport in asthma. J Clin Invest 126, 2367-2371 (2016).
10. Lachowicz-Scroggins, M. E., et al. Abnormalities in MUC5AC and MUC5B Protein in Airway Mucus in Asthma. Am J Respir Crit Care Med 194, 1296-1299 (2016).
11. Wesener, D. A., et al. Recognition of microbial glycans by human intelectin-1. Nat Struct Mol Biol 22, 603-610 (2015).
12. Tsuji, S., et al. Human intelectin is a novel soluble lectin that recognizes galactofuranose in carbohydrate chains of bacterial cell wall. J Biol Chem 276, 23456-23463 (2001).
13. Kerr, S. C., et al. Intelectin-1 is a prominent protein constituent of pathologic mucus associated with eosinophilic airway inflammation in asthma. Am J Respir Crit Care Med 189, 1005-1007 (2014).
14. Pemberton, A. D., Rose-Zerilli, M. J., Holloway, J. W., Gray, R. D. & Holgate, S. T. A single-nucleotide polymorphism in intelectin 1 is associated with increased asthma risk. J Allergy Clin Immunol 122, 1033-1034 (2008).
15. Barrett, J. C., et al. Genome-wide association defines more than 30 distinct susceptibility loci for Crohn's disease. Nat Genet 40, 955-962 (2008).
16. Franke, A., et al. Genome-wide meta-analysis increases to 71 the number of confirmed Crohn's disease susceptibility loci. Nat Genet 42, 1118-1125 (2010).
17. Travaglini, K. J., et al. A molecular cell atlas of the human lung from single-cell RNA sequencing. Nature 587, 619-625 (2020).
18. Xu, G., Forstner, G. G. & Forstner, J. F. Interaction of heparin with synthetic peptides corresponding to the C-terminal domain of intestinal mucins. Glycoconj J 13, 81-90 (1996).
19. Xu, G., Bell, S. L., McCool, D. & Forstner, J. F. The cationic C-terminus of rat Muc2 facilitates dimer formation post translationally and is subsequently removed by furin. Eur J Biochem 267, 2998-3004 (2000).
20. Sajuthi, S. P., et al. Type 2 and interferon inflammation strongly regulate SARS-CoV-2 related gene expression in the airway epithelium. bioRxiv (2020).
21. Sajuthi, S. P., et al. Nasal airway transcriptome-wide association study of asthma reveals genetically driven mucus pathobiology. Nat Commun 13, 1632 (2022).
22. Kesimer, M., et al. Airway Mucin Concentration as a Marker of Chronic Bronchitis. N Engl J Med 377, 911-922 (2017).
23. Nonnecke, E. B., et al. Human intelectin-1 (ITLN1) genetic variation and intestinal expression. Sci Rep 11, 12889 (2021).
24. Gu, N., et al. Intelectin is required for IL-13-induced monocyte chemotactic protein-1 and -3 expression in lung epithelial cells and promotes allergic airway inflammation. Am J Physiol Lung Cell Mol Physiol 298, L290-296 (2010).
25. Kuperman, D. A., et al. Dissecting asthma using focused transgenic modeling and functional genomics. J Allergy Clin Immunol 116, 305-311 (2005).
26. Watanabe, T., et al. Expression of intelectin-1 in bronchial epithelial cells of asthma is correlated with T-helper 2 (Type-2) related parameters and its function. Allergy Asthma Clin Immunol 13, 35 (2017).
27. Yuan, S., et al. Oxidation increases mucin polymer cross-links to stiffen airway mucus gels. Sci Transl Med 7, 276ra227 (2015).
28. Tang, M., et al. Mucus Plugs Persist in Asthma, and Changes in Mucus Plugs Associate with Changes in Airflow over Time. Am J Respir Crit Care Med 205, 1036-1045 (2022).
29. Neophytou, A. M., et al. Air Pollution and Lung Function in Minority Youth with Asthma in the GALA II (Genes-Environments and Admixture in Latino Americans) and SAGE II (Study of African Americans, Asthma, Genes, and Environments) Studies. Am J Respir Crit Care Med 193, 1271-1280 (2016).
30. Nishimura, K. K., et al. Early-life air pollution and asthma risk in minority children. The GALA II and SAGE II studies. Am J Respir Crit Care Med 188, 309-318 (2013).
31. Reynolds, S. D., et al. Airway Progenitor Clone Formation Is Enhanced by Y-27632-Dependent Changes in the Transcriptome. Am J Respir Cell Mol Biol 55, 323-336 (2016).
32. Everman, J. L., Rios, C. & Seibold, M. A. Utilization of Air-Liquid Interface Cultures as an In Vitro Model to Assess Primary Airway Epithelial Cell Responses to the Type 2 Cytokine Interleukin-13. Methods Mol Biol 1799, 419-432 (2018).
33. Chu, H. W., et al. CRISPR-Cas9-mediated gene knockout in primary human airway epithelial cells reveals a proinflammatory role for MUC18. Gene Ther 22, 822-829 (2015).
34. Everman, J. L., Rios, C. & Seibold, M. A. Primary Airway Epithelial Cell Gene Editing Using CRISPR-Cas9. Methods Mol Biol 1706, 267-292 (2018).
35. Allan D. B.; Caswell, T. K. N. C. v. d. W., C. M.; and Verweij, R. W. soft-matter/trackpy: Trackpy v0.5.0 (v0.5.0). (2021).
36. Kuznetsova, A., Brockhoff, P. B. & Christensen, R. H. B. lmerTest Package: Tests in Linear Mixed Effects Models. Journal of Statistical Software 82, 1-26 (2017).
37. Chioccioli, M., et al. Quantitative High-Speed Video Profiling Discriminates between DNAH11 and HYDIN Variants of Primary Ciliary Dyskinesia. Am J Respir Crit Care Med 199, 1436-1438 (2019).
38. Feriani, L., et al. Assessing the Collective Dynamics of Motile Cilia in Cultures of Human Airway Cells by Multiscale DDM. Biophys J 113, 109-119 (2017).
39. Royle, L., et al. Glycan structures of ocular surface mucins in man, rabbit and dog display species differences. Glycoconj J 25, 763-773 (2008).
40. Traag, V. A., Waltman, L. & van Eck, N.J. From Louvain to Leiden: guaranteeing well-connected communities. Sci Rep 9, 5233 (2019).
41. Jiang, H., Lei, R., Ding, S. W. & Zhu, S. Skewer: a fast and accurate adapter trimmer for next-generation sequencing paired-end reads. BMC Bioinformatics 15, 182 (2014).
42. Wu, T. D. & Nacu, S. Fast and SNP-tolerant detection of complex variants and splicing in short reads. Bioinformatics 26, 873-881 (2010).
43. Anders, S., Pyl, P. T. & Huber, W. HTSeq—a Python framework to work with high-throughput sequencing data. Bioinformatics 31, 166-169 (2015).
44. Bray, N. L., Pimentel, H., Melsted, P. & Pachter, L. Near-optimal probabilistic RNA-seq quantification. Nat Biotechnol 34, 525-527 (2016).
45. Love, MI., Huber, W. & Anders, S. Moderated estimation of fold change and dispersion for RNA-seq data with DESeq2. Genome Biol 15, 550 (2014).
46. Consortium GT, L. D., Coordinating Center—Analysis Working G, Statistical Methods groups-Analysis Working G, Enhancing Gg, Fund NIHC, et al. Genetic effects on gene expression across human tissues. Nature. 2017; 550(7675):204-13.
47. Robinson, M. D., McCarthy, D. J. & Smyth, G. K. edgeR: a Bioconductor package for differential expression analysis of digital gene expression data. Bioinformatics 26, 139-140 (2010).
48. McCarthy, D. J., Chen, Y. & Smyth, G. K. Differential expression analysis of multifactor RNA-Seq experiments with respect to biological variation. Nucleic Acids Res 40, 4288-4297 (2012).
49. Stegle, O., Parts, L., Piipari, M., Winn, J. & Durbin, R. Using probabilistic estimation of expression residuals (PEER) to obtain increased power and interpretability of gene expression analyses. Nat Protoc 7, 500-507 (2012).
50. Alexander, D. H., Novembre, J. & Lange, K. Fast model-based estimation of ancestry in unrelated individuals. Genome Res 19, 1655-1664 (2009).
51. Ongen, H., Buil, A., Brown, A. A., Dermitzakis, E. T. & Delaneau, O. Fast and efficient QTL mapper for thousands of molecular phenotypes. Bioinformatics 32, 1479-1485 (2016).
52. Delaneau, O., et al. A complete tool set for molecular QTL discovery and analysis. Nat Commun 8, 15452 (2017).
53. Gershman, N. H., Wong, H. H., Liu, J. T., Mahlmeister, M. J. & Fahy, J.V. Comparison of two methods of collecting induced sputum in asthmatic subjects. Eur Respir J 9, 2448-2453 (1996).
54. Peters, M. C., et al. Refractory airway type 2 inflammation in a large subgroup of asthmatic patients treated with inhaled corticosteroids. J Allergy Clin Immunol 143, 104-113 e114 (2019).
55. Peters, M. C., et al. Measures of gene expression in sputum cells can identify TH2-high and TH2-low subtypes of asthma. J Allergy Clin Immunol 133, 388-394 (2014).
56. Cardet, J. C., et al. Clinical and molecular implications of RGS2 promoter genetic variation in severe asthma. J Allergy Clin Immunol (2022).
57. Kim, D., Langmead, B. & Salzberg, S. L. HISAT: a fast spliced aligner with low memory requirements. Nat Methods 12, 357-360 (2015).
58. Radicioni, G., et al. Airway mucin MUC5AC and MUC5B concentrations and the initiation and progression of chronic obstructive pulmonary disease: an analysis of the SPIROMICS cohort. Lancet Respir Med 9, 1241-1254 (2021).
59. Yu, Q., et al. Benchmarking the Orbitrap Tribrid Eclipse for Next Generation Multiplexed Proteomics. Anal Chem 92, 6478-6485 (2020).
60. Conant, D., et al. Inference of CRISPR Edits from Sanger Trace Data. CRISPR J 5, 123-130 (2022).

What is claimed is:

1. A method of reducing mucus obstruction in a subject in need thereof, comprising administering to the subject an intelectin-1 (ITLN-1) inhibitor.

2. The method of claim 1, wherein the subject is a T2-high asthmatic subject.

3. The method of claim 1, wherein the mucus obstruction is mucus plugging.

4. The method of claim 1, wherein the ITLN-1 inhibitor reduces mucus viscosity.

5. The method of claim 1, wherein the ITLN-1 inhibitor increases muco-ciliary clearance.

6. The method of claim 1, wherein the ITLN-1 inhibitor is a chemical inhibitor of ITLN-1 or a biological inhibitor of ITLN-1.

7. The method of claim 1, wherein the ITLN-1 inhibitor is an anti-intelectin molecule or an anti-intelectin antibody.

8. The method of claim 1, wherein the ITLN-1 inhibitor is in a pharmaceutical composition.

9. A pharmaceutical composition comprising an ITLN-1 inhibitor.

10. The pharmaceutical composition of claim 9, wherein the ITLN-1 inhibitor reduces mucus obstruction in a T2-high asthmatic subject.

11. The pharmaceutical composition of claim 10, wherein the mucus obstruction is mucus plugging.

12. The pharmaceutical composition of claim 10, wherein the ITLN-1 inhibitor reduces mucus viscosity.

13. The pharmaceutical composition of claim 10, wherein the ITLN-1 inhibitor increases muco-ciliary clearance.

14. The pharmaceutical composition of claim 9, wherein the ITLN-1 inhibitor is a chemical inhibitor of ITLN-1 or a biological inhibitor of ITLN-1.

15. The pharmaceutical composition of claim 9, wherein the inhibitor is an anti-intelectin molecule or an anti-intelectin antibody.

16. A pharmaceutical composition comprising an ITLN-1 inhibitor, for use in reducing mucus obstruction in a subject in need thereof.

17. The pharmaceutical composition of claim 16, wherein the subject is a T2-high asthmatic subject.

18. The pharmaceutical composition of claim 16, wherein the mucus obstruction is mucus plugging.

19. The pharmaceutical composition of claim 16, wherein the ITLN-1 inhibitor reduces mucus viscosity or increases muco-ciliary clearance.

20. The pharmaceutical composition of claim 16, wherein the ITLN-1 inhibitor is a chemical inhibitor of ITLN-1, a biological inhibitor of ITLN-1, an anti-intelectin molecule or an anti-intelectin antibody.
